(19) United States
(12) Patent Application Publication
(10) Pub. No.: US 2013/0225623 A1
(43) Pub. Date: Aug. 29, 2013

(54) Methods of Treating Psychiatric or Neurological Disorders with MGLUR Antagonists

(71) Applicants: Joseph Buxbaum, New York, NY (US); Takeshi Sakurai, New York, NY (US); Ozlem (Bozdagi) Gunal, New York, NY (US)

(72) Inventors: Joseph Buxbaum, New York, NY (US); Takeshi Sakurai, New York, NY (US); Ozlem (Bozdagi) Gunal, New York, NY (US)

(73) Assignee: MOUNT SINAI SCHOOL OF MEDICINE, New York, NY (US)

(21) Appl. No.: 13/504,847

(22) PCT Filed: Oct. 27, 2010
(86) PCT No.: PCT/US10/54260

§ 371 Date: Jul. 3, 2012

(30) Foreign Application Priority Data
Oct. 27, 2010 (US) 61255340

(51) Int. Cl. A61K 31/44; A61K 31/197

(52) U.S. Cl. CPC A61K 31/44; A61K 31/197
USPC 514/277

(57) ABSTRACT

Methods for treating a psychiatric or neurological disease or disorder using combinations of Group 1 mGluR antagonists are disclosed. In certain aspects, these methods include the treatment of a patient having a neurological or psychiatric disease or disorder associated with a CYFIP1 gene change.

### CROSS REFERENCE TO RELATED APPLICATIONS

This application claims priority under 35 U.S.C. §119(e) of provisional application 61/255,340, filed Oct. 27, 2009, which is hereby incorporated herein by reference in its entirety.

### STATEMENT REGARDING FEDERALLY SPONSORED RESEARCH

This invention was made in part in the course of research supported by National Institutes of Health, Department of Health and Human Services, Grant No. U54 MH066673. The U.S. government has certain rights in the invention.

### FIELD OF THE INVENTION

The present invention is related to methods for treating a psychiatric or neurological disease or disorder using a composition containing an mGluR5 antagonist in combination with an mGluR1 antagonist. In certain aspects, the methods of the invention are directed to treating a patient having a disease or disorder associated with a cytoplasmic FMRP interacting protein 1 (CYFIP1) gene change. In some aspects, the disease or disorder is Fragile X syndrome (FXS), wherein the CYFIP1-binding protein called Fragile X mental retardation protein (FMRP) is directly involved, an autism spectrum disorder, schizophrenia, Prader-Willi syndrome, or Angelman syndrome.

### BACKGROUND OF THE INVENTION

In the mammalian central nervous system (CNS), the transmission of nerve impulses at the synapse is controlled by the interaction between a neurotransmitter released by a sending neuron and a surface receptor on a receiving neuron, causing excitation or inhibition of this receiving neuron. The ability of the synapse to change in strength is known as synaptic plasticity. L-Glutamate, the most abundant neurotransmitter in the CNS, mediates the major excitatory pathway in mammals, and is referred to as an excitatory amino acid (EAA). Importantly, L-glutamate stimulates protein synthesis in neurons, which is required for several different forms of synaptic plasticity.

The receptors that respond to glutamate are called excitatory amino acid receptors (EAA receptors). See Watkins & Evans, Annual Reviews in Pharmacology and Toxicology, 21: 165 (1981), Monaghan, Bridges, and Cotman, Annual Reviews in Pharmacology and Toxicology, 29: 365 (1989); Watkins, Krogsgaard-Larsen, and Honore, Transactions in Pharmaceutical Science, 11: 25 (1990).

EAA receptors are classified into two general types. Receptors that are directly coupled to the opening of cation channels in the cell membrane of the neurons are termed “ionotropic.” This type of receptor has been subdivided into at least three classes, which are defined by the depolarizing actions of the selective agonists N-methyl-D-aspartate (NMDA), α-amino-3-hydroxy-5-methylisoxazole-4-propionic acid (AMPA), and kainic acid (KA). Five kainate receptors, classified as either high affinity (KA1 and KA2) or low affinity (GluR5, GluR6 and GluR7) kainate receptors have been identified. See Bleakman et al., Molecular Pharmacology, 1996, Vol. 49, No. 4, pp. 581-585.

The second general type of receptor is the G-protein or second messenger-linked “metabotropic” EAA receptor. This second type is a highly heterogeneous family of glutamate receptors that are linked to multiple second messenger systems and are called metabotropic glutamate receptors (mGluRs). Based on their amino acid sequence homology, agonist pharmacology, and coupling to transduction mechanisms, the 8 presently known mGluR sub-types are classified into three groups.

Group I receptors (mGluR1 and mGluR5) have been shown to be coupled to stimulation of phospholipase C resulting in phosphoinositide hydrolysis and elevation of intracellular Ca⁺⁺ levels, and, in some expression systems, to modulation of ion channels, such as K⁺ channels, Ca⁺ channels, non-selective cation channels, or NMDA receptors. Group II receptors (mGluR2 and mGluR3) and Group III receptors (mGluRs 4, 6, 7, and 8) are negatively coupled to adenylcyclase and have been shown to couple to inhibition of cAMP formation when heterologously expressed in mammalian cells, and to G-protein-activated inward rectifying potassium channels in Xenopus oocytes and in unipolar brush cells in the cerebellum. Besides mGluR6, which is essentially only expressed in the retina, the mGluR5 are widely expressed throughout the central nervous system.

Both types of EAA receptors appear not only to mediate normal synaptic transmission along excitatory pathways, but also participate in the modification of synaptic connections during development and throughout life. See, Schoepp, Bockaert, and Sladeczek, Trends in Pharmacological Science, 11: 508 (1990); McDonald and Johnson, Brain Research Reviews, 15: 41 (1990).

Protein synthesis at the synapse is stimulated by the major excitatory neurotransmitter glutamate via group I mGluRs. Long-term depression (LTD) is dependent on mGluR5 and most importantly requires the rapid synthesis of new proteins in dendrites (Huber et al., 2000). Several studies have demonstrated that activation of mGluR5 with either synaptic stimulation or the selective agonist R,S-dihydroxyphenylglycine (DHPG) induces LTD of synaptic responses in area CA1 of the rat hippocampus [Fitzjohn S M, et al. Neuropharmacology. 1999 October; 38(10):1577-83; Kemp N, Bashir Z I. Neuropharmacology. 1999 April; 38(4):495-504; Huber K M, et al. J Neurophysiol. 2001 July; 86(1):321-5]. This LTD mechanism provides clues to the function of glutamate or activity-induced stimulation of local dendritic protein synthesis.

Control of L-glutamate induced protein synthesis is a critical mechanism for modulating long-term changes in neural circuits and resultant behavioral changes (Costa-Mattioli et al., 2009). CYFIP1 is a critical part of the complex that regulates mRNA transport and translation at the synapse (Napoli et al., 2008). Cytoplasmic FMRP interacting protein 1 (CYFIP1) is part of a complex with FMRP (Fragile X mental retardation protein) and disruption of the levels of this complex, as occurs in Fragile X syndrome leads to neurological and psychiatric conditions.

Although it was known that mGluR activation can stimulate protein synthesis, including that of FMRP, the functional role of this mechanism was unknown until recently. The molecular basis for FXS was discovered when it was found that FXS patients have an expansion in the 5′ untranslated region of the FMR1 gene, which results in transcriptional silencing. [Chakrabarti L, Davies K E. Curr Opin Neurol. 1997 April; 10(2):142-7.] The loss of the FMR1 gene product, FMRP, is responsible for the Fragile X phenotype [Pieretti M, et al. Cell. 1991 Aug. 23; 66(4):817-22.; Verheij C, et al. Nature. 1993 Jun. 24; 363(6431):722-4]. CYFIP1 has a direct role in a process that is disrupted in FXS, as CYFIP1 interacts with FMRP (Schenck et al., 2001). The focus on altered functioning of mGluR has been called the mGluR hypothesis in FXS and is reflected in enhanced hippocampal (mGluR-dependent) LTD (“mGluR-LTD”) in Fmr1 knockout mice with the later stages of LTD no longer showing the same requirement for protein synthesis (as the normal control on protein synthesis mediated by FMRP is lost). Recently, it has been shown that CYFIP1 can directly bind to the translation initiation factor eIF4E and, like FMRP, negatively regulates FMRP target mRNAs (Napoli et al., 2008). Stimulation of neurons was shown to cause the dissociation of CYFIP1 from eIF4E at synapses, resulting in protein synthesis, thus providing a mechanism for the activity-dependent regulation of translation seen with FMR1 and CYFIP1.

By virtue of its interaction with FMRP, changes in the CYFIP1 gene (e.g., mutations or copy number variations (CNVs) such as deletions or duplications) can be a factor causing FXS. FXS patients who also have a CYFIP1 gene change are likely to have a more severe phenotype than those without a CYFIP1 gene change. Other psychiatric or neurological disorders can also be associated with CNVs in regions that include CYFIP1 that can affect normal synaptic plasticity. Thus, there is a need in the art for methods to regulate abnormal EAA receptor activity for the treatment of psychiatric disorders and neurological conditions. The present invention provides such methods.

### SUMMARY OF THE INVENTION

In certain aspects, the present invention provides methods for treating a patient having a disease or disorder associated with a cytoplasmic FMRP interacting protein 1 (CYFIP1) gene change, which includes the steps of: identifying a patient in need of such treatment and administering to said patient an effective amount for treating said disease or disorder of a composition including an mGluR5 antagonist and an mGluR1 antagonist.

In certain aspects, the present invention provides a medicament and/or other composition comprising an mGluR5 antagonist and/or an mGluR1 antagonist for use in treating a disease or disorder associated with a cytoplasmic FMRP interacting protein 1 (CYFIP1) gene change, wherein the medicament and/or other composition is administered to a patient in an effective amount for treating said disease or disorder.

In certain other aspects, the invention provides methods for treating a neurological or psychiatric disease or disorder, which involves administering to a patient in need of such treatment an effective amount for treating said disease or disorder of a composition including an mGluR5 antagonist and an mGluR1 antagonist.

In certain other aspects, the invention provides a medicament and/or other composition comprising an mGluR5 antagonist and or an mGluR1 antagonist for use in treating a neurological or psychiatric disease or disorder, by administering the medicament and/or other composition to a patient in an effective amount for treating said disease or disorder.

In some aspects, the neurological or psychiatric disease is associated with a copy number variation in the 15q11.2 gene region.

In some embodiments of the invention, a method for treating a patient having a disease or disorder associated with a copy number variation (CNV) in the 15q11.2 gene region is provided, which involves the steps of: identifying a patient in need of such treatment and administering to said patient an effective amount for treating said disease or disorder of a composition including an mGluR5 antagonist in combination with an mGluR1 antagonist.

In some embodiments, the invention provides a medicament and/or other composition comprising an mGluR5 antagonist and or an mGluR1 antagonist for use in treatment of a disease or disorder with a copy number variation (CNV) in the 15q11.2 gene region, by administering the medicament and/or other composition to a patient in an effective amount for treating said disease or disorder.

In any of the embodiments of the invention, the disease or disorder that may be treated by the methods and compositions of the present invention is selected from the group consisting of an autism spectrum diagnosis (ASD), Fragile X syndrome, schizophrenia, Prader-Willi syndrome, and Angelman syndrome. Further, in any of the embodiments of the invention, the patient may be a human.

In some embodiments of the invention, an mGluR1 antagonist is a member selected from the group consisting of: LY367385, A 841720, LY 456236 hydrochloride, Bay 36-7620 and CPCCOEt.

In some aspects of the invention, the mGlur5 antagonist is 2-methyl-6-(phenylethynyl)-pyridine (MPEP) and the mGluR1 antagonist is LY367385.

In other embodiments, an mGluR5 antagonist of the invention is a member selected from the group consisting of 2-methyl-6-(phenylethynyl)-pyridine (MPEP), (E)-6-methyl-2-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3 SR,4aRS,6SR,8aRS-6-(((4-carboxy)phenyl)methyl)-1,2,3,4,4a,5,6,7,8,8a-deca hydroisoquinoline-3-carboxylic acid, [N-(3-chlorophenyl)-N′-(4,5-dihydro-1-methyl-4-oxo-1H-imidazole-2-yl)urea] (Fenobam), 3-((2-Methyl-1,3-thiazol-4-yl)ethynyl)pyridine hydrochloride (MTEP hydrochloride), and 3S,4aR,6S,8aR-6-(((4-carboxy)-phenyl)methyl)-1,2,3,4,4a,5,6,7,8,8a-decahydroisoquinoline-3-carboxylic acid.

In certain aspects, an mGluR5 antagonist of the invention is administered in a dose ranging from between about 0.0001 mg to about 100 mg per kilogram of body weight per day. In yet other aspects, an mGluR1 antagonist of the invention is administered in a dose ranging from between about 0.0001 mg to about 100 mg per kilogram of body weight per day.

In some embodiments, a CYFIP1 gene change is a member selected from the group consisting of a CYFIP1 duplication, a CYFIP1 deletion, a mutation in the CYFIP1 gene resulting in increased expression of CYFIP1, and a mutation in the CYFIP1 gene resulting in decreased expression of CYFIP1.

In yet other embodiments of the invention, a pharmaceutical composition containing (a) an effective amount for treating a disease or disorder associated with a CYFIP1 gene change of a composition including an mGluR5 antagonist and an mGluR1 antagonist; and (b) a pharmaceutically acceptable carrier or diluent.

In still other embodiments, the present invention provides a pharmaceutical composition containing (a) an mGluR5 antagonist and an mGluR1 antagonist in an effective amount for treating a disease or disorder associated with a copy number variation (CNV) in the 15q11.2 gene region; and (b) a pharmaceutically acceptable carrier or diluent. In certain aspects, any one of the pharmaceutical compositions of the invention is provided as a pharmaceutical dosage form. In some aspects the pharmaceutical dosage form is a tablet or capsule.

In certain aspects, the compositions of the invention are administered intravenously.

In some embodiments of the invention, the pharmaceutical composition is used to treat a disease or disorder selected from the group consisting of an autism spectrum diagnosis (ASD), Fragile X syndrome, schizophrenia, Prader-Willi syndrome, and Angelman syndrome.

These and other aspects of the present invention will be apparent to those of ordinary skill in the art in light of the present specification, claims and drawings.

### BRIEF DESCRIPTION OF THE DRAWINGS

FIG. 1 shows a schematic of the 15q11.2 region.

FIG. 2A shows pedigrees from three families with a 15q11.2 deletion (del) or duplication (dup). Squares indicate male family member, circles indicate female family members. Shapes representing family members identified as having an ASD are shaded.

FIG. 2B and FIG. 2C show results obtained from multiplex ligation-dependent amplification (MLPA) carried out across 15q11.2. Examples from the patient with a 15q11.2 deletion (Family 1) (FIG. 2B) and a patient with a 15q11.2 duplication (Family 3) (FIG. 2C) are shown.

FIG. 3A shows the genomic structure of CYFIP1 to scale with larger horizontal boxes representing exons, and the first (ATG) and last (Stop) coding exons indicated. The site of the gene-trap insertion (identified as LTR-flanked TRAPPING CASSETTE) in intron 1 (5′ to the first coding exon), is indicated.

FIG. 3B shows an immunoblot of brain samples from wild-type and Cyfip1 heterozygous mice using anti-Cyfip1 antibody.

FIG. 3C shows the relative mRNA expression of the indicated genes in brain lysates from wild-type (Wt) and Cyfip1 heterozygous (Het) mice. ***, P=0.004.

FIG. 4 shows the field EPSP slope in hippocampal slices from wild type (Wt) and Cyfip1 heterozygotes (het) at increasing stimulus intensity (mA).

FIG. 5A shows average field EPSP slope normalized to baseline, before and after LTP induction. LTP is induced with 100 Hz tetanic stimulation for 1 second in hippocampal slices from wild-type (Wt) or Cyfip1 heterozygous (Het) mice. Onset of stimulation is indicated by arrows.

FIG. 5B shows average field EPSP slope normalized to baseline, before and after LTP induction. LTP is induced with high frequency stimulation (4 trains of 100 Hz, 1 s stimulation separated by 5 min) in hippocampal slices from wild-type (Wt) or Cyfip1 heterozygous (Het) mice. Onset of stimulation is indicated by arrows.

FIG. 5C shows average field EPSP slope normalized to baseline, before and after LTP induction. LTP is induced with threshold levels of theta burst stimulation in hippocampal slices from wild-type (Wt) or Cyfip1 heterozygous (Het) mice. Onset of stimulation is indicated by arrows.

FIG. 6 shows average field EPSP slope normalized to baseline, before and after LTD induction. LTD is induced with paired-pulse low frequency stimulation (PP-LFS) in hippocampal slices from wild type (Wt) and Cyfip1 heterozygous (Het) mice. Onset of stimulation is indicated by the arrow. Inset: Representative EPSP traces were recorded before stimulation (1) or 60 min after stimulation (2) in wild-type and heterozygous animals (calibration bars represent 10 msec on the horizontal axis and 0.5 mV on the vertical axis).

FIG. 7A-7B are field EPSP slopes of LTD induced by paired-pulse low frequency stimulation (PP-LFS) in wild-type (A) or Cyfip1 heterozygous (B) mice, in the absence (open symbols) or presence (filled symbols) of the protein synthesis inhibitor cycloheximide (Cyclohex, 60 μM) (C,D). Onset of stimulation is indicated by arrows.

FIG. 7C-7D are field EPSP slopes of LTP induced by high frequency stimulation (HFS; 4 trains of 100 Hz, 1 s stimulation separated by 5 min), in wild-type (C) or Cyfip1 heterozygous (D) mice, in the absence (open symbols) or presence (filled symbols) of the protein synthesis inhibitor cycloheximide (Cyclohex, 60 μM). In each panel there were 6 animals per group. Onset of stimulation is indicated by arrows.

FIG. 8A shows field EPSP slopes of LTD induced by DHPG (50 μM for 5 minutes, indicated by the short horizontal bar) in hippocampal slices from wild-type (Wt) and Cyfip1 heterozygous (Het) mice.

FIGS. 8B and 8C show two field EPSP slopes of LTD induced by DHPG (50 μM for 5 minutes, indicated by the short horizontal bar) in hippocampal slices from wild-type (Wt, 8B) and Cyfip1 heterozygous (Het, 8C) mice in the absence (open symbols) or presence (closed symbols) of cycloheximide (Cyclohex, 60 μM, indicated by the long horizontal bar).

FIG. 9 shows field EPSP slopes of LTD induced by DHPG (50 μM, indicated by the short horizontal bar) in hippocampal slices from wild-type (WT, upper panel) or Cyfip1 heterozygous (Het, lower panel) mice, in the absence (open symbols) or presence (filled symbols) of rapamycin (20 nM, indicated by the long horizontal bar).

FIG. 10 shows field EPSP slopes of LTD induced by DHPG (50 μM, indicated by the short horizontal bar) in hippocampal slices from wild-type (open symbols) or Cyfip1 heterozygous (closed symbols) mice, the latter in the absence (circles) or presence (squares) of both MPEP (10 μM) and LY367385 (indicated by the long horizontal bar).

FIG. 11 shows the results of an inhibitory avoidance protocol for wild-type (wt) and Cyfip1 heterozygous mice (Het) at training and 6 h, 24 h and 48 h following training.

### DETAILED DESCRIPTION OF THE INVENTION

The present invention provides methods for treating a neurological or psychiatric disease or disorder using mGluR antagonists. In one aspect, the methods of the present invention are directed to treating a patient with a disease or disorder associated with a cytoplasmic FMRP interacting protein 1 (CYFIP1) gene change. As described in detail in the present Examples, the specific combination of mGluR1 and mGluR5 antagonists is surprisingly more beneficial than use of either antagonist alone for the treatment of disorders associated with a CYFIP1 gene change.

As used herein, a “CYFIP1 gene change” includes CYFIP1 mutations and gene dosage abnormalities, also called copy number variations (CNVs), caused, e.g., by deletion or duplication.

The term “gene dosage” refers to the number of copies of a particular gene present in a subject. The location of a human gene is given as the chromosome number and the region of the chromosome (locus) where the gene is found. For example, “15q11.2” refers to the 11.2 region of the q arm on human chromosome 15.

The human and murine amino acid and nucleic acid sequences of CYFIP1/Cyfip1 are known and have been described. The CYFIP1 (human) nucleic acid sequences have Genbank® Accession numbers NM_{—}014608.2 (SEQ ID NO: 1) and NM_{—}001033028.1 (SEQ ID NO: 2); and the amino acid sequences have Accession numbers NP_{—}055423.1 (SEQ ID NO: 3) and NP_{—}001028200.1 (SEQ ID NO: 4). The Cyfip1 (murine) nucleic acid sequence has Genbank® Accession number NM_{—}011370.1 (SEQ ID NO: 5) and the amino acid sequence has Accession number NP_{—}035500.1 (SEQ ID NO: 6). [See, Feingold et al. Proc. Natl. Acad. Sci. U.S.A. 99 (26), 16899-16903 (2002); Carninci, P. and Hayashizaki, Y. Genome Res. 10 (10), 1617-1630 (2000); Shibata, K. et al. Genome Res. 10 (11), 1757-1771 (2000); Kawai, J. et al. Nature 409 (6821), 685-690 (2001); Okazaki, Y., et al. Nature 420 (6915), 563-573 (2002); Carninci, P. et al. Science 309 (5740), 1559-1563 (2005).]

Genomic imbalances, including CNVs, have been shown to be etiologically significant in many patients with autism spectrum diagnoses (ASDs) (Cook and Scherer, 2008). These genomic imbalances include those that are inherited or de novo and those that are recurrent and not recurrent. Recurrent genomic imbalances typically arise due to non-allelic homologous recombination (NHAR) mediated by low-copy repeats (LCR) (more recently also referred to as segmental duplications) (Gu and Lupski, 2008).

Some recurrent genomic imbalances can increase risk for diverse psychiatric disorders, including ASDs, schizophrenia, attention deficit/hyperactivity disorder, and obsessive-compulsive disorder. For example, it has been known for some time that CNVs in the 22q11 region are associated with schizophrenia and autism, while more recently, additional rarer CNVs have been shown to be associated with these same two disorders (O'Donovan et al., 2008; Burbach and van der Zwaag, 2009).

It is presently discovered that CNVs in the 15q11.2 region are correlated with autism spectrum diagnoses (ASDs). The 15q11.2 region includes a minimal 0.3 Mb region that encompasses at least 4 genes, including TUBGCP5, CYFIP1, NIPA2, and NIPA1 [Chai et al., 2003]. It is also presently discovered that CYFIP1 gene changes are associated with ASDs, and that CYFIP1 is an important target for the treatment of ASDs, as well as other psychiatric diseases or disorders characterized by CNVs in the 15q11.2 region.

ASDs are a spectrum of psychological conditions characterized by widespread abnormalities of social interactions and communication, as well as severely restricted interests and/or highly repetitive behavior. Autism forms the core of the ASDs. The defining characteristics of ASDs are qualitative impairments of social communication and interaction, along with restricted and repetitive activities and interests. Individual symptoms occur in the general population and appear not to associate highly, without a sharp line separating pathological severity from common traits. Other aspects of ASDs, such as atypical eating, are also common but are not essential for diagnosis; they can affect the individual or the family. Most recent reviews tend to estimate a prevalence of 1-2 per 1,000 for autism and close to 6 per 1,000 for ASD; (Newschaffer C J, et al. (2007) Annu Rev Public Health 28: 235-58.) because of inadequate data, these numbers may underestimate ASD's true prevalence. See, Caronna E B, et al. (2008) Arch Dis Child 93 (6): 518-23.

A number of different treatments for autism have been developed. Many of the treatments, however, address the symptoms of the disease rather than the causes. For example, therapies ranging from behavioral therapies to psychopharmacology have been employed in the treatment of autism. Although some clinical symptoms may be lessened by these treatments, modest improvement, at best, has been demonstrated in only a minor fraction of the cases. Only a small percentage of autistic persons become able to function as self-sufficient adults. Thus, new therapeutic treatment methods for ASDs such as autism are needed. The present invention provides such methods.

As described below, schizophrenia, Prader-Willi syndrome (PWS) and Angelman syndrome (AS) are also associated with CNVs in the 15q11.2 region. In certain aspects, these psychiatric conditions can also benefit from the methods of the present invention.

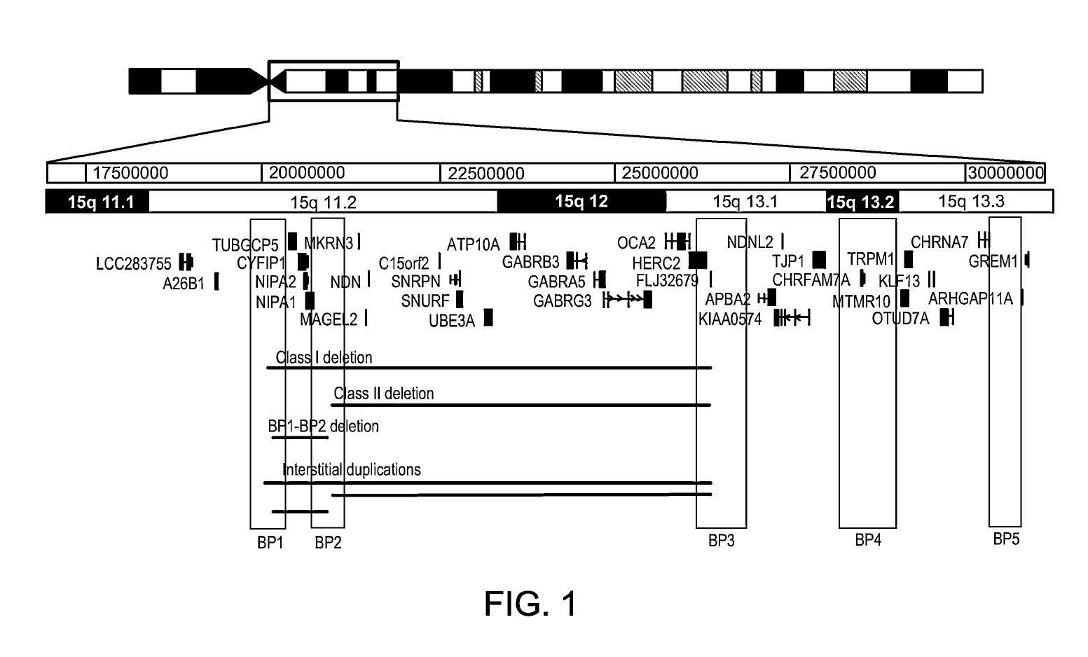

Schizophrenia is a psychiatric diagnosis that describes a mental disorder characterized by abnormalities in the perception or expression of reality. Distortions in perception may affect all five senses, including sight, hearing, taste, smell and touch, but most commonly manifest as auditory hallucinations, paranoid or bizarre delusions, or disorganized speech and thinking with significant social or occupational dysfunction. Onset of symptoms typically occurs in young adulthood, with approximately 0.4-0.6% of the population affected. Recently, a recurrent CNV was identified to increase risk for schizophrenia by 2-4 fold in two large studies involving the 15q11.2 region (Stefansson et al., 2008; Kirov et al., 2008) (see FIG. 1). The CNV identified by these studies includes a minimal 0.3 Mb region that encompasses CYFIP1. Thus, the methods of the present invention, which are useful for treating patients having a psychiatric disorder and a CYFIP1 gene change, are useful for the treatment of schizophrenia.

Similarly, PWS and AS are also associated with CNVs in regions that include the 15q11.2 region (Murthy et al., 2007). PWS and AS are caused by a loss of paternally (in PWS) or maternally (in AS) expressed genes in the 15q11-q13 region (reviewed in Horsthemke and Wagstaff, 2008). While AS has been directly attributed to loss of maternal expression of the UBE3A gene (reviewed in Lalande and Calciano, 2007), the genes responsible for PWS are less clear cut, although recent evidence indicates the loss of the HBII-85 small nucleolar RNA (snoRNA) cluster in 15q11-13 is a major cause of the phenotype (Sahoo et al., 2008). In both syndromes, the loss can be due to a deletion in 15q11-13. The proximal breakpoint for such deletions can be at one of two loci, termed BP1 and BP2 (Chai et al., 2003) (see FIG. 1), resulting, respectively, in a longer type I or a shorter type II deletion. The BP1-BP2 interval corresponds to the 15q11.2 region identified in schizophrenia and illustrated in FIG. 1.

In a study of patients with AS, comparing Type I to Type II deletions indicated that the Type I deletion was associated with an increased severity of speech impairments as well as a delay in sitting without support (Varela et al., 2004). Furthermore, there was significantly increased likelihood of having an ASD in individuals with AS arising from a Type I deletion (Sahoo et al., 2006). These larger deletions were associated with lower cognitive scores, lower expressive language scores, and more severe seizure risk (Sahoo et al., 2006).

Taken together, the data indicate that in both PWS and AS the larger deletion is associated with a more severe (or broader) phenotype and implicate the BP1-BP2 genes in this. Behavioral outcomes in PWS have been correlated with expression of the genes in the BP1-BP2 interval, with higher levels of expression associated with better outcomes (Bittel et al., 2006). CYFIP1 is present in this BP1-BP2 interval, therefore making PWS and AS disorders that can be treated according to the methods of the present invention.

In certain embodiments, the methods of the present invention are also useful for the treatment of Fragile X syndrome (FXS). FXS is the most common inherited form of mental retardation, affecting 1 in 1500 men and 1 in 2500 women (de Vries, et al., 1993). Patients with FXS can exhibit many neurological deficiencies or conditions, including moderate to severe mental retardation (IQ=30-70), seizures (e.g., benign childhood epilepsy, temporal lobe epilepsy), visual spatial defects, anxiety, learning difficulties and certain characteristics of autism.

In the present Examples, long-term depression (LTD) is measured to analyze synaptic plasticity. LTD is the weakening of a neuronal synapse that lasts from hours to days. Thus, a measurement of increased LTD indicates weakened synaptic signaling. It results from either strong synaptic stimulation (as occurs in the cerebellar Purkinje cells) or persistent weak synaptic stimulation (as in the hippocampus). LTD is thought to result from changes in postsynaptic receptor density, although changes in presynaptic release may also play a role. Hippocampal/cortical LTD can be dependent of NMDA receptors, mGluRs or endocannabinoids. LTD is distinct from synaptic depotentiation, which is the reversal of long-term potentiation (LTP). LTD is a novel reduction in synaptic strength—specifically, an activity-dependent reduction in the excitatory post-synaptic potential compared to the baseline level.

It has been suggested that an LTD-like mechanism could be responsible for elimination or pruning of inappropriate synapses which are formed during early periods of postnatal development. [Colman H, et al. Science. 1997 Jan. 17; 275(5298):356-61; Bear M F, Rittenhouse C D, J Neurobiol. 1999 October; 41(1):83-91.] Recent evidence supports this hypothesis. Treatment of hippocampal neuronal cultures with the group I mGluR agonist, DHPG, results in a long-term decrease in the surface expression of AMPA-subtype glutamate receptors (AMPAR), the receptors responsible for synaptic transmission at excitatory synapses. Like LTD, the long-term decrease in the AMPAR surface expression is dependent on protein synthesis (Snyder, et al., 2001). Preliminary data also indicate a concomitant reduction in the number of presynaptic terminals after DHPG treatment. Together, these results indicate that activation of mGluR5 results in decreases in synaptic strength most likely mediated by a reduction or elimination in the number of excitatory synapses. This synapse elimination process may contribute to the formation of appropriate synaptic connections during development as well as in the storage of memories in the adult.

LTP is the opposing process to LTD. LTP is the long-lasting improvement in communication between two neurons that results from stimulating them simultaneously. LTP is commonly divided into three phases that occur sequentially: short-term potentiation, early LTP, and late LTP.

The early (E-LTP) and late (L-LTP) phases of LTP are each characterized by a series of three events: induction, maintenance, and expression. Induction is the process by which a short-lived signal triggers that phase of LTP to begin. Maintenance corresponds to the persistent biochemical changes that occur in response to the induction of that phase. Expression entails the long-lasting cellular changes that result from activation of the maintenance signal. See, Lynch M (2004) Physiol Rev 84 (1): 87-136; Huang Y, Kandel E (1994) Learn Mem 1 (1): 74-82; Sweatt J (1999) Learn Mem 6 (5): 399-416; Agranoff, Bernard W.; Siegel, George J. (1999). Basic neurochemistry: molecular, cellular, and medical aspects. Philadelphia: Lippincott-Raven. pp. 326; Malenka R, Bear M (2004) Neuron 44 (1): 5-21; Malinow R (2003). Philos Trans R Soc Lond B Biol Sci 358 (1432): 707-14; Frey U, et al. (1 Jan. 1996). J Physiol 490 (Pt 3) (Pt 3): 703-11; Frey U, et al. H (1988). Brain Res 452 (1-2): 57-65; Kovacs et al. (2007). PNAS 104 (11): 4700-5; Lynch M (2004). Physiol Rev 84 (1): 87-136.

In the present Examples, it is demonstrated that CYFIP1 plays an integral part in the LTD mechanism that is critical to normal synaptic plasticity. Disruption of normal synaptic plasticity, such as e.g., increased LTD, is associated with neurological and psychiatric disorders.

As used herein, the term “subject” or “individual” refers to an animal, preferably a mammal (e.g., rodent, such as mouse). In particular, the term refers to humans.

As used herein, the term “about” or “approximately” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

An “effective amount” refers to the amount of a compound including an mGluR antagonist that is effective, upon single or multiple dose administration to a patient, in treating the patient suffering from the named disorder.

The term “ED₅₀” means the dose of a drug that produces 50% of its maximum response or effect.

The term “IC₅₀” means the concentration of a drug which inhibits an activity or property by 50%, e.g., by reducing the frequency of a condition, such as cell death, by 50%, by reducing binding of a competitor peptide to a protein by 50% or by reducing the level of an activity by 50%.

The term “LD₅₀” means the dose of a drug that is lethal in 50% of test subjects.

“Composition” indicates a combination of multiple substances into an aggregate mixture.

The term “statistically significant” as used herein means that the obtained results are not likely to be due to chance fluctuations at the specified level of probability. The two most commonly specified levels of significance are 0.05 (P=0.05) and 0.01 (P=0.01). The level of significance equal to 0.05 and 0.01 means that the probability of error is 5 out of 100 and 1 out of 100, respectively.

Exemplary mGluR Antagonists

The present invention relates to the use of antagonists of Group I mGluRs, such as antagonists of mGluR5 and mGluR1, for treating FXS, schizophrenia, Prader-Willi syndrome, Angelman syndrome and ASDs, including autism. The present invention is also directed to the use of antagonists of Group I mGluRs, such as antagonists of mGluR5 and mGluR1, for treating a disease or disorder associated with a CYFIP1 gene change. In particularly preferred embodiments of the invention, mGluR5 and mGluR1 antagonists are used in combination.

In a particular embodiment, children with FXS, schizophrenia, Prader-Willi syndrome, Angelman syndrome or an ASD, including autism, can be treated with Group I mGluR antagonists. Preferably treatment is with a combination of mGluR1 and mGluR5 antagonists. The children can be treated during infancy (between about 0 to about 1 year of life), childhood (the period of life between infancy and puberty) and during puberty (between about 8 years of life to about 18 years of life). In still another embodiment, the methods of the invention can be used to treat adults (greater than about 18 years of life) having FXS, schizophrenia, Prader-Willi syndrome, Angelman syndrome or an ASD, including autism.

An “agonist” is a molecule which activates a certain type of receptor. For example, glutamate molecules act as agonists when they excite EM receptors. An example of an agonist of the present invention is DHPG, which induces mGluR5-dependent long-term depression (LTD).

By contrast, an “antagonist” is a molecule which prevents or reduces the effects exerted by an agonist on a receptor. As used herein, the term “antagonist” includes antagonists and inverse agonists (also known as “reverse agonists”). An inverse agonist is an agent which binds to the same receptor binding-site as an agonist for that receptor and reverses the constitutive activity of the receptor. Whereas an antagonist blocks the ability of an agonist to activate a receptor and needs the presence of agonist to block the activity, an inverse agonist binds to a receptor and reverses the action of the receptor, in the absence of agonist. An inverse agonist is also known as a reverse agonist. An example of an antagonist of the present invention is 2-methyl-6-(phenylethynyl)-pyridine (MPEP), which is an mGluR5 antagonist and inhibits the ability of DHPG to induce mGluR5-mediated LTD. An example of an inverse agonist that may be used as an antagonist in the present invention is Fenobam [N-(3-chlorophenyl)-N′-(4,5-dihydro-1-methyl-4-oxo-1H-imidazole-2-yl)urea], which is a noncompetitive mGluR5 antagonist with inverse agonist activity. See, Porter et al. (2005) J Pharmacol Exp Ther. November; 315(2):711-21.

As used herein, the term “mGluR antagonist” includes group I mGluR antagonists, such as mGluR1 and mGluR5 antagonists.

In certain embodiments, preferred antagonists are those that provide a reduction of activation by the ligand or reveres the activity of the receptor by at least 10%, and more preferably at least 20%, at least 30%, at least 40%, at least 50%, at least 60%, at least 70%, at least 80%, at least 90%, at least 95%, or even at least 99% at a concentration of the antagonist, for example, of 1 μg/ml, 10 μg/ml, 100 μg/ml, 500 μg/ml, 1 mg/ml, 10 mg/ml, or 100 mg/ml. The percentage antagonism represents the percentage decrease in activity of mGluR, e.g., mGluR5, in a comparison of assays in the presence and absence of the antagonist. Any combination of the above mentioned degrees of percentage antagonism and concentration of antagonist may be used to define an antagonist of the invention, with greater antagonism at lower concentrations being preferred.

An antagonist for use in the invention may be a relatively non-specific antagonist that is an antagonist of mGluRs in general. Preferably, however, an antagonist selectively antagonizes mGluR5 and/or mGluR1. Even more preferably, an antagonist used in the invention is a selective antagonist of either mGluR5 or mGluR1. With respect to mGluR5, a selective antagonist of mGluR5 is one that antagonizes mGluR5, but antagonizes other mGluRs only weakly or substantially not at all, or at least antagonizes other mGluRs with an EC₅₀ at least 10 or even 100 or 1000 times greater than the EC₅₀ at which it antagonizes mGluR5. A selective antagonist of mGluR1 is one that antagonizes mGluR1, but antagonizes other mGluRs only weakly or substantially not at all, or at least antagonizes other mGluRs with an EC₅₀ at least 10 or even 100 or 1000 times greater than the EC₅₀ at which it antagonizes mGluR1. Most preferred antagonists are those which can selectively antagonize their target at low concentrations, for example, those that cause a level of antagonism of 50% or greater at a concentration of 100 μg/ml or less.

Exemplary mGluR5 antagonists include, without limitation, 2-methyl-6-(phenylethynyl)-pyridine (MPEP), (E)-6-methyl-2-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3 SR,4aRS,6SR,8aRS-6-(((4-carboxy)phenyl)methyl)-1,2,3,4,4a,5,6,7,8,8a-decahydroisoquinoline-3-carboxylic acid, 3-((2-Methyl-1,3-thiazol-4-yl)ethynyl)pyridine hydrochloride (MTEP hydrochloride) and 3S,4aR,6S,8aR-6-(((4-carboxy)-phenyl)methyl)-1,2,3,4,4a,5,6,7,8,8a-decahydroisoquinoline-3-carboxylic acid, and their pharmaceutically acceptable salts, analogues and derivatives thereof.

Antagonists of mGluR5 are also described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, U.S. Pat. No. 6,218,385, U.S. Pat. No. 5,672,592, U.S. Pat. No. 5,795,877, U.S. Pat. No. 5,863,536, U.S. Pat. No. 5,880,112, U.S. Pat. No. 5,902,817, U.S. Pat. No. 5,962,521, U.S. Pat. No. 5,968,915, U.S. Pat. No. 6,054,444 and U.S. Pat. No. 5,977,090.

For example, different classes of mGluR5 antagonists are described in WO 01/08705 (pp. 3-7), WO 99/44639 (pp. 3-11), and WO 98/34907 (pp. 3-20).

Examples of antagonists of mGluR1 include but are not limited to LY367385, A 841720, LY 456236 hydrochloride, Bay 36-7620, and CPCCOEt, and their pharmaceutically acceptable salts, analogues and derivatives thereof.

Antagonists, including inverse agonists, for use in the present invention are commercially available, e.g., from Tocris Bioscience (Ellisville, Mo.).

In certain embodiments, an mGluR1 antagonist and an mGluR5 antagonist of the invention can be administered together in one composition or in two different compositions, which are administered simultaneously or sequentially (to the same or different sites). In a specific embodiment of the invention, the mGluR5 antagonist MPEP is used in combination with the mGluR1 antagonist LY367385 for the treatment of a neurological or psychiatric condition.

Another class of mGluR1 antagonists, antisense oligonucleotides, is described in WO 01/05963. Antisense oligonucleotides to mGluR5 can be prepared by analogy and used to selectively antagonize mGluR5, as desired.

Also contemplated by the present invention are additional small molecule inhibitors of mGluR1 and mGluR5. Diverse libraries of small molecule inhibitors can be generated. The compounds of the present invention, particularly libraries of variants having various representative classes of substituents, are amenable to combinatorial chemistry and other parallel synthesis schemes (see, for example, PCT WO 94/08051). The result is that large libraries of related compounds, e.g., a variegated library of potential mGluR antagonists, can be screened rapidly in high-throughput assays to identify potential lead compounds, as well as to refine the specificity, toxicity, and/or cytotoxic-kinetic profile of a lead compound.

A variety of techniques are available in the art for generating combinatorial libraries of small organic molecules such as the subject antagonists. See, for example, Blondelle et al. (1995) Trends Anal. Chem. 14: 83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116: 2661: Kerr et al. (1993) JACS 115: 252; PCT publications WO92/10092, WO93/09668 and WO91/07087; and the Lerner et al. PCT publication WO93/20242). Accordingly, a variety of libraries on the order of about 100 to 1,000,000 or more diversomers of the subject antagonists can be synthesized and screened for a particular activity or property. These methods are described in detail in U.S. Pat. No. 6,916,821.

The above described mGluR antagonists are meant to serve as representative examples only. It should be understood that any agent or small compound that is useful as an mGluR1 or mGluR5 antagonist is contemplated for use in the methods and compositions of the present invention.

### Methods and Assays

### Experimental Synaptic Plasticity

To measure synaptic plasticity in vitro, hippocampal slices may be assayed for electrophysiology. Measurements including input/output function, paired-pulse facilitation, or various forms of LTP may be evaluated. Input/output relationship is a measurement of the synaptic function and reflects the synaptic response to the number of axons activated by a given stimulus. A shift in the relationship indicates a change in the excitability. Long-term potentiation (LTP) may be induced by either a high-frequency stimulus (four trains of 100 Hz, 1 s stimulation separated by 5 min), a threshold level of theta-burst stimulation (TBS) (5 bursts of four pulses at 100 Hz separated by 200 ms, (Lauterborn et al., 2007), or a single 100 Hz stimulation. Different LTP induction protocols may activate distinct signaling cascades that generate LTP with different expression mechanisms. [See, Malenka R C, Nicoll R A, Science 1999, 285:1870; Blundon J A and Zakharenko S S, Neuroscientist 2008; 14:598.] To induce an mGluR-dependent LTD, Schaffer collaterals may be stimulated by a paired-pulse low-frequency stimulation (PP-LFS, 1 Hz for 20 min; 50 ms interstimulus interval (Huber et al., 2000). LTD may also be induced using the agonist DHPG.

In the above assays, the excitatory postsynaptic potential (EPSP) of a synapse is determined. An EPSP is a temporary depolarization of postsynaptic membrane potential caused by the flow of positively charged ions into the postsynaptic cell as a result of opening of ligand-sensitive channels. EPSPs are the opposite of inhibitory postsynaptic potentials (IPSPs), which usually result from the flow of negative ions into the cell or positive ions out of the cell.

A postsynaptic potential is defined as excitatory if it makes it easier for the neuron to fire an action potential. EPSPs can also result from a decrease in outgoing positive charges, while IPSPs are sometimes caused by an increase in positive charge outflow. The flow of ions that causes an EPSP is an excitatory postsynaptic current (EPSC). EPSPs, like IPSPs, are graded (i.e. they have an additive effect). When multiple EPSPs occur on a single patch of postsynaptic membrane, their combined effect is the sum of the individual EPSPs. Larger EPSPs result in greater membrane depolarization and thus increase the likelihood that the postsynaptic cell reaches the threshold for firing an action potential.

EPSPs may be recorded using intracellular electrodes. The extracellular signal from a single neuron is extremely small and thus next to impossible to record. However, in some areas of the brain, such as the hippocampus, neurons are arranged in such a way that they all receive synaptic inputs in the same area. Because these neurons are in the same orientation, the extracellular signals from synaptic excitation don't cancel out, but rather add up to give a signal that can easily be recorded with a field electrode. This extracellular signal recorded from a population of neurons is the field potential.

In studies of hippocampal LTP, figures may be given showing the field EPSP (fEPSP) in stratum radiatum of CA1 in response to Schaffer collateral stimulation. This is the signal seen by an extracellular electrode placed in the layer of apical dendrites of CA1 pyramidal neurons. The Schaffer collaterals make excitatory synapses onto these dendrites, and so when they are activated, there is a current sink in stratum radiatum: the field EPSP. The voltage deflection recorded during a field EPSP is negative-going, while an intracellularly recorded EPSP is positive-going. This difference is due to the relative flow of ions (primarily the sodium ion) into the cell, which, in the case of the field EPSP is away from the electrode, while for an intracellular EPSPs it is towards the electrode.

After a field EPSP, the extracellular electrode may record another change in electrical potential named the population spike which corresponds to the population of cells firing action potentials (spiking). In other regions than CA1 of the hippocampus, the field EPSP may be far more complex and harder to interpret as the source and sinks are far less defined. In regions such as the striatum neurotransmitters such as dopamine, acetylcholine, GABA and others may also be released and further complicate the interpretation.

### Multiplex Ligation-Dependent Probe Amplification (MLPA)

As described in the present Examples, CNVs in 15q11.2 were confirmed by multiplex ligation-dependent probe amplification (MLPA).

MLPA is a variation of polymerase chain reaction (PCR) that permits multiple targets to be amplified with only a single primer pair. Each probe consists of a two oligonucleotides which recognize adjacent target sites on the DNA. One probe oligonucleotide contains the sequence recognized by the forward primer, the other the sequence recognized by the reverse primer. Only when both probe oligonucleotides are hybridized to their respective targets, can they be ligated into a complete probe. The advantage of splitting the probe into two parts is that only the ligated oligonucleotides, but not the unbound probe oligonucleotides, are amplified. If the probes were not split in this way, the primer sequences at either end would cause the probes to be amplified regardless of their hybridization to the template DNA, and the amplification product would not be dependent on the number of target sites present in the sample DNA.

Each complete probe has a unique length, so that its resulting amplicons can be separated and identified by (capillary) electrophoresis. Since the forward primer used for probe amplification is fluorescently labeled, each amplicon generates a fluorescent peak which can be detected by a capillary sequencer. Comparing the peak pattern obtained on a given sample with that obtained on various reference samples, the relative quantity of each amplicon can be determined. This ratio is a measure for the ratio in which the target sequence is present in the sample DNA.

MLPA can successfully and easily determine the relative copy number of all exons within a gene simultaneously with high sensitivity. An important use of MLPA is to determine relative ploidy (i.e. to determine whether CNVs are present). For example, probes may be designed to target various regions of chromosome 21 of a human cell. The signal strengths of the probes are compared with those obtained from a reference DNA sample known to have two copies of the chromosome. If an extra copy is present in the test sample, the signals are expected to be 1.5 times the intensities of the respective probes from the reference. If only one copy is present the proportion is expected to be 0.5. If the sample has two copies, the relative probe strengths are expected to be equal.

SNP Arrays

As described in the present examples, genome-wide single-nucleotide polymorphism (SNP) arrays were used to screen for CNVs in patients with ASDs.

A single-nucleotide polymorphism (SNP) is a DNA sequence variation occurring when a single nucleotide—A, T, C, or G—in the genome (or other shared sequence) differs between members of a species (or between paired chromosomes in an individual). Almost all common SNPs have only two alleles.

Within a population, SNPs can be assigned a minor allele frequency—the lowest allele frequency at a locus that is observed in a particular population. This is simply the lesser of the two allele frequencies for single-nucleotide polymorphisms. There are variations between human populations, so a SNP allele that is common in one geographical or ethnic group may be much rarer in another. Single nucleotide may be changed (substitution), removed (deletions) or added (insertion) to polynucleotide sequence. Ins/del SNP may shift translational frame.

Single-nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are nonsynonymous. A nonsynonymous change may either be missense or nonsense, where a missense change results in a different amino acid, while a nonsense change results in a premature stop codon. SNPs that are not in protein-coding regions may still have consequences for gene splicing, transcription factor binding, or the sequence of non-coding RNA.

SNPs are often found to be the etiology of many human diseases and are becoming of particular interest in pharmacogenetics. SNPs can be detected by various techniques such as dynamic allele-specific hybridization (DASH), which takes advantage of the differences in the melting temperature in DNA that results from the instability of mismatched base pairs, (see, Howell W., et al. (1999) Nat Biotechnol. 17(1):87-88); by molecular beacons, which make use of a specifically engineered single-stranded oligonucleotide probe (see, Abravaya et al. (2003) Clin Chem Lab Med. 41:468-474); and by SNP arrays (Rapley R., Harbron S. (Eds.) (2004) Molecular Analysis and Genome Discovery. Chichester. John Wiley & Sons Ltd.).

In high density oligonucleotide SNP arrays, hundreds of thousands of probes are arrayed on a small chip, allowing for many SNPs to be interrogated simultaneously. Because SNP alleles only differ in one nucleotide and because it is difficult to achieve optimal hybridization conditions for all probes on the array, the target DNA has the potential to hybridize to mismatched probes. This is addressed somewhat by using several redundant probes to interrogate each SNP. Probes are designed to have the SNP site in several different locations as well as containing mismatches to the SNP allele. By comparing the differential amount of hybridization of the target DNA to each of these redundant probes, it is possible to determine specific homozygous and heterozygous alleles (Rapley & Harbron 2004). The Affymetrix® Human SNP 5.0 GeneChip performs a genome-wide assay that can genotype over 500,000 human SNPs (Affymetrix (2007) Genome-Wide Human SNP Array 5.0. [online] Address: http://www.affymetrix.com/products/arrays/specific/genome_wide/genome_wide_snp_{—}5.affx).

It is to be understood that BP1-BP2 CNVs and/or CYFIP1 gene changes in a patient may be determined by any method known in the art. Non-limiting examples of identifying BP1-BP2 CNVs and/or CYFIP1 gene changes are RT-PCR, MLPA, or SNPs. Any suitable sample from a patient may be used. Non-limiting examples include a tissue sample or blood.

### Human ASD Diagnosis

### Developmental Milestones

When diagnosing autism or another ASD, developmental milestones of a patient are compared to normal ranges for developmental milestones, which are summarized as follows (adapted from the CDC website (http://www.cdc.gov/ncbddd/actearly/milestones/index.html) and from CARING FOR YOUR BABY AND YOUNG CHILD: BIRTH TO AGE 5 by Steven Shelov, Robert E. Hannermann, © 1991, 1993, 1998, 2004 by the American Academy of Pediatrics.):

Social/Emotional

3-Months

Social smile; enjoys playing with others; imitates

7-Months

Enjoys social play; interested in mirror images; responds to expression of emotion; appears joyful often

1-Year

Shy or anxious with strangers; cries when mother or father leaves; shows preferences for certain people and toys; may be fearful in some situations; prefers mother and/or regular caregiver over all others; repeats sounds or gestures for attention; extends arm or leg to help when being dressed

2-Years

Imitates behavior of others; more aware as separate from others; more excited about company of other children; demonstrates increasing independence; begins to show defiant behavior; separation anxiety increases toward midyear then fades

3-Years

Can take turns in games; understands concept of “mine” and “his/hers”; expresses affection openly; expresses wide range of emotions; separates easily from parents; objects to major changes in routine

4-Years

Interested in new experiences; cooperates with other children; plays “Mom” or “Dad”; increasingly inventive in fantasy play; dresses and undresses; negotiates solutions to conflicts; more independent; imagines that many unfamiliar images may be “monsters”; views self as a whole person involving body, mind, and feeling; often cannot tell the difference between fantasy and reality

5-Years

Wants to please friends; wants to be like their friends; more likely to agree to rules; likes to sing, dance, and act; shows more independence and may even visit a next-door neighbor alone; aware of gender; able to distinguish fantasy from reality; sometimes demanding, sometimes eagerly cooperative

Cognitive Milestones

7-Months

Finds partially hidden object; explores with hands/mouth; struggles to get objects out of reach

1-Year

Explores objects in many different ways (shaking, banging, throwing, dropping); finds hidden objects easily; looks at correct picture when the image is named; imitates gestures; begins to use objects correctly (drinking from cup, brushing hair, dialing phone, listening to receiver)

2-Years

Finds objects even when hidden under two or three covers; begins to sort by shapes and colors; begins make-believe play

3-Years

Makes mechanical toys work; matches an object in her hand or room to a picture in a book; plays make-believe with dolls, animals, and people; sorts objects by shape and color; completes puzzles with three or four pieces; understands concept of “two”

4-Years

Correctly names some colors; understands the concept of counting and may know a few numbers; tries to solve problems from a single point of view; begins to have a clearer sense of time; follows three-part commands; recalls parts of a story; understands the concepts of “same” and “different”

5-Years

Can count 10 or more objects; correctly names at least four colors; better understands the concept of time; knows about things used every day in the home (money, food, appliances)

Motor

3-Months

Raises head; supports upper body with arms while lying on stomach; opens/shuts hands; brings hand to mouth

7-Months

Rolls both ways; sits; supports weight on legs; reaches with one hand; transfers object from hand to hand

1-Year

Crawls forward on belly; pulls self up to stand; walks holding on to furniture; stands momentarily without support; may walk two or three steps without support; uses pincer grasp; bangs two objects together; puts objects into container; takes objects out of container; tries to imitate scribbling

2-Years

Walks alone; pulls toys while walking; begins to run; stands on tiptoe; kicks a ball; climbs onto and down from furniture unassisted; walks up and down stairs with support; scribbles on his or her own; turns over container to pour out contents; builds tower of four blocks or more; favors one hand

3-Years

Climbs well; walks up and down stairs with alternating feet with support; runs easily; pedals tricycle; makes up-and-down, side-to-side, and circular lines with pencil or crayon; turns book pages one at a time; builds a tower of more than six blocks; holds a pencil in writing position; screws and unscrews jar lids and bolts; turns rotating handles

4-Years

Hops and stands on one foot up to five seconds; goes upstairs and downstairs without support; kicks ball forward; throws ball overhand; catches bounced ball most of the time; moves forward and backward with agility; copies square shapes; draws a person with two to four body parts; uses scissors; draws circles and squares; begins to copy some capital letters

5-Years

Stands on one foot for 10 seconds or longer; hops, somersaults; swings, climbs; may be able to skip; copies triangle and other shapes; draws person with body; prints some letters; dresses and undresses without help; uses fork, spoon, and (sometimes) a table knife; usually cares for own toilet needs

Languag e

3-Months

Smiles at sound of voice; babbles; imitates sound; turns head toward sound

7-Months

Responds to name; begins to respond to “no”; can detect emotion in voice; responds to sound by making sounds; babbles chains of sound; uses voice to express joy and displeasure

1-Year

Pays attention to speech; responds to simple verbal requests; responds to “no”; uses simple gestures (shaking head for “no”); babbles with inflection (changes in tone); says “dada” and “mama”; uses exclamations (“oh-oh!”); tries to imitate words

2-Years

Points to object or picture when it's named; recognizes names of familiar people, objects, and body parts; says several single words (by 15 to 18 months); uses simple phrases (by 18 to 24 months); uses 2- to 4-word sentences; follows simple instructions; repeats words overheard in conversation

3-Years

Follows two- or three-part command; recognizes and identifies almost all common objects and pictures; understands most sentences; understands placement in space (“on,” “in,” “under”); Uses 4- to 5-word sentences; can say name, age, and sex; uses pronouns (I, you, me, we, they) and some plurals (cars, dogs, cats); strangers can understand most of their words

4-Years

Mastered some basic rules of grammar; speaks in sentences of five to six words; speaks clearly enough for strangers to understand; Tells stories

5-Years

Recalls part of a story; speaks sentences of more than five words; uses future tense; tells longer stories; says name and address

Vision

3-Months

Watches faces; follows moving objects; recognizes faces; uses hand/eyes in coordination

7-Months

Develops full color vision; distance vision matures; tracking of moving objects improves

### Apgar Scores

The Apgar score provides a convenient shorthand for reporting the status of the newborn infant and the response to resuscitation. It is described in detail in “The Apgar Score”; American Academy of Pediatrics; Volume 117, Number 4, April 2006: 1444-1447.

### ADOS and ADI

Autism Diagnostic Interview-Revised (ADI-R) is described in detailed by Lord, C., et al. (1994). Autism diagnostic interview-revised: A revised version of a diagnostic interview for caregivers of individuals with possible pervasive developmental disorders. Journal of Autism and Developmental Disorders, 24(5), 659-685). The ADI-R assesses communication, social impairment, and compulsive behaviors in addition to early developmental history, motor functioning, and general behaviors. It is a semi-structured psychiatric interview designed for the study of autism and related disorders and is typically administered to the patient's primary caretaker/family member. In children 2 years and older, the ADI-R demonstrates good validity in diagnosing autistic disorder (Lord et al., 1994).

Autism Diagnostic Observation Schedule-Generic (ADOS-G) is described in detail by Lord C, Rutter M, Goode S et al. (1989). “Autism diagnostic observation schedule: a standardized observation of communicative and social behavior”. J Autism Dev Disord 19 (2): 185-212. The ADOS-G assesses functioning in each of the three core symptom domains (communication, compulsivity, social impairment) as well as associated features of autism. The ADOS-G involves a standardized observation protocol of social and communicative behavior in children, adolescents, and adults. This tool is commonly used in research, specifically paired with the ADI-R to complement a thorough diagnosis.

### Murine Model of ASDs

### Physical and Motor Development

In the murine model of ASDs, the condition is assessed based on physical, reflex and locomotor landmarks, as described below:

Physical, Reflex and Locomotor
Landmarks in a Murine ASD Model

| | Average Age for | |
|---|---|---|
| | Response (days) | Range (days) |
| Physical landmarks | | |
| Pinnae detachment | 15 | 10-20 |
| Eye opening | 13 | 7-17 |
| Incisor eruption | 7 | 5-10 |
| Fur development | 11 | 3-15 |
| Reflexes | | |
| Surface righting | 5 | 1-10 |
| Air righting | 18 | 16-21 |
| Negative geotaxis | 7 | 3-15 |
| Cliff avoidance | 8 | 2-12 |
| Visual placing | 15 | 11-18 |
| Forelimb/hindlimb placing^{a} | 5 | 1-10 |
| Vibrissa placing response | 9 | 5-15 |
| Auditory startle | 15 | 11-21 |
| Tactile startle | 15 | 3-20 |
| Crossed extensor reflex^{a} | 3 | 1-10 |
| Rooting reflex^{a} | 2 | 1-15 |
| Grasp reflex^{a} | 7 | 3-15 |
| Bar holding | 14 | 10-21 |
| Level screen test | 8 | 5-15 |
| Vertical screen test | 19 | 15-21 |
| Locomotor behavior | | |
| Elevation of head | 12 | 9-21 |
| Elevation of forelimbs | 7 | 5-15 |
| and shoulders | | |
| Pivoting^{a} | 7 | 2-17 |
| Crawling^{a} | 11 | 7-16 |
| Walking | 16 | 12-21 |

^{a}Behavior either disappears or reduces to <0 in frequency.

Detailed methods for mouse models of ASD are described in Current Protocols in Neuroscience, section 8.18.1-15, by Charles Heyser, 2003, and also in What's wrong with my mouse? Behavioral phenotyping of transgenic and knockout mice, 2^{nd} edition, 2007, by Jacqueline Crawley.

### Cyfip1 Mice

As described in the present Examples, since FMRP is known to interact with CYFIP1 in a complex that regulates protein synthesis in dendrites, the 15q11.2 region in ASDs was examined and the function of the CYFIP1 gene was determined using mice with a disruption of the Cyfip1 gene. Mice with a disruption in Cyfip1 were generated from gene-trapped embryonic stem (ES) cells.

Mice were developed from an Omnibank (Lexicon) embryonic stem (ES) cell line that was produced by mutagenesis with a gene trap insertional vector. Briefly, an ES clone was identified that has a trapping cassette inserted into intron 1 of the Cyfip1 gene (note that the start ATG is in exon 2). A mouse line was established from the ES cells in the 129SvEvBrd strain. The ES cells were injected into C57BL6/J mice (The Jackson Laboratory, Bar Harbor, Me.) to obtain chimeric mice. Chimeric mice were mated with C57BL6 mice from Taconic (Hudson, N.Y.) to obtain heterozygotes and were subsequently maintained on the C57BL6 background.

Gene trapping is a high-throughput approach that is used to introduce insertional mutations across the mammalian genome. It is performed with gene trap vectors whose principal element is a gene trapping cassette consisting of a promoterless reporter gene and/or selectable genetic marker flanked by an upstream 3′ splice site (splice acceptor; SA) and a downstream transcriptional termination sequence (polyadenylation sequence; polyA). When inserted into an intron of an expressed gene, the gene trap cassette is transcribed from the endogenous promoter of that gene in the form of a fusion transcript in which the exon(s) upstream of the insertion site is spliced in frame to the reporter/selectable marker gene. Since transcription is terminated prematurely at the inserted polyadenylation site, the processed fusion transcript encodes a truncated and non-functional version of the cellular protein and the reporter/selectable marker. Thus, gene traps simultaneously inactivate and report the expression of the trapped gene at the insertion site, and provide a DNA tag (gene trap sequence tag, GTST) for the rapid identification of the disrupted gene.

### Immunoblotting

Methods for immunoblotting, also known as Western blotting, are well known in the art. In the present invention, Cyfip1 protein expression is determined using a monoclonal antibody specific for Cyfip1 (Synaptic Systems GmbH, Germany).

### Protein Synthesis Inhibition

In the present Examples, protein synthesis is preferably inhibited using cycloheximide. Examples of other protein synthesis inhibitors that may be used include, but are not limited to anisomycin and cycloheximide (also emetine and puromycin).

Compositions Containing mGluR Antagonists

In certain aspects, the present invention provides compositions containing one or more group 1 mGluR antagonists. Preferably, the group 1 mGluR antagonists are selected from mGluR1 and mGluR5 antagonists. Still more preferably, mGluR1 and mGluR5 antagonists are used in combination.

In one aspect of the invention, a patient in need of such treatment is administered a composition containing an mGluR5 antagonist in combination with an mGluR1 antagonist of the invention. In other aspects, an mGluR1 antagonist and an mGluR5 antagonist may be administered in separate compositions, at the same site or at a different site of administration, and at the same or at different times.

The compositions of the invention can be formulated for administration in any convenient way for use in human or veterinary medicine. The mGluR antagonists of the invention may be incorporated into liposomes, microemulsions, micelles, unilamellar or multilamellar vesicles, erythrocyte ghosts or spheroblasts. In one embodiment, the antagonists of the invention can be delivered in one or more vesicles, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

In yet another embodiment, the mGluR antagonists of the invention can be delivered in a controlled release form. For example, the mGluR antagonists may be administered in a polymer matrix such as poly (lactide-co-glycolide) (PLGA), in a microsphere or liposome implanted subcutaneously, or by another mode of delivery (see, Cao et al., 1999, Biomaterials, February;20(4):329-39). Another aspect of delivery includes the suspension of the compositions of the invention in an alginate hydrogel.

The term “therapeutically effective” when applied to a dose or an amount refers to that quantity of a compound or pharmaceutical composition that is sufficient to result in a desired activity upon administration to a mammal in need thereof. As used herein, the term “therapeutically effective amount/dose” refers to the amount/dose of a pharmaceutical composition of the invention that is suitable for treating a patient or subject having an autoimmune disease. In certain embodiments of the invention the patient or subject may be a mammal. In certain embodiments, the mammal may be a human.

Pharmaceutical formulations of the present invention can also include veterinary compositions, e.g., pharmaceutical preparations of the mGluR antagonists suitable for veterinary uses, e.g., for the treatment of livestock or domestic animals, e.g., dogs.

When formulated in a pharmaceutical composition, the compositions of the present invention can be admixed with a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicles with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Alternatively, the carrier can be a solid dosage from carrier, including but not limited to one or more of a binder (for compressed pills), a tablet, an encapsulating agent, a flavorant, and a colorant. Suitable pharmaceutical carriers are described in “Remington's Pharmaceutical Sciences” by E.W. Martin.

The optimum concentration of the active ingredient(s) in the chosen carrier, or biologically acceptable medium, can be determined empirically, according to procedures well known to medicinal chemists. As used herein, “biologically acceptable medium” includes any and all solvents, dispersion media, and the like which may be an appropriate carrier for the desired route of administration of the pharmaceutical preparation. The use of such media for pharmaceutically active substances is known in the art. Except insofar as any conventional media or agent is incompatible with the activity of the mGluR antagonists, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

### Administration

The compositions and formulations of the present invention can be administered topically, parenterally, orally, by inhalation, as a suppository, or by other methods known in the art. The term “parenteral” includes injection (for example, intravenous, epidural, intrathecal, intramuscular, intraluminal, intratracheal or subcutaneous). The compositions of the present invention may also be administered using a transdermal patch.

Administration of the compositions of the invention may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

The mGluR antagonists compositions described herein can be used to treat or prevent psychiatric or neurological disorders. As used herein, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving the disease, i.e., causing regression of the state, disorder or condition or at least one of its clinical or subclinical symptoms.

The benefit to an individual to be treated is either statistically significant or at least perceptible to the patient or to the physician.

It will be appreciated that the amount of the mGluR antagonists of the invention required for use in treatment will vary with the route of administration, the nature of the condition for which treatment is required, and the age, body weight and condition of the patient, and will be ultimately at the discretion of the attendant physician or veterinarian. These compositions will typically contain an effective amount of the compositions of the invention, alone or in combination with an effective amount of any other active material, e.g., those described above. Preliminary doses can be determined according to animal tests, and the scaling of dosages for human administration can be performed according to art-accepted practices.

Keeping the above description in mind, typical dosages of mGluR1 and mGluR5 antagonists of the invention may range from about 0.0001 mg to about 100 mg per kilogram of body weight per day. In certain embodiments, a patient may receive, for example, 1 mg per day of each antagonist intravenously.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### EXAMPLES

The present invention is described further below in working examples which are intended to further describe the invention without limiting the scope therein.

In the examples below, the following materials and methods were used.

Subjects

Complete details of a study initiated by L. Alison McInnes (LAM) on the genetics of ASDs in the Central Valley of Costa Rica, including recruitment and assessment, have been described (McInnes et al. 2005; McInnes et al. 2007). The study was approved under the guidelines of the Ministry of Health of Costa Rica, the Ethical Committee of the National Children's Hospital in San Jose (Hospital Nacional de Niños, or HNN), and the Institutional Review Board at Mount Sinai School of Medicine in accordance with the Declaration of Helsinki

All parents of the patients provided written informed consent for both participation and publication. Families of individuals with a clinical ASD diagnosis or a possible ASD diagnosis contacted the HNN, or were contacted by the Costa Rican research team, and after expressing interest in the study, were formally asked to participate using established informed consent criteria. All interviews and exams took place in the Neurodevelopmental Unit of the HNN, where parents were interviewed by an experienced pediatrician using the Autism Diagnostic Interview-Revised (ADI-R) and the Autism Diagnostic Observation Schedule (ADOS) was administered to subjects, with both of these assessments videotaped for independent scoring by the best estimator (ERM).

IQ tests appropriate for the age and level of verbal communication of the subjects were administered, as was the Vineland Adaptive Behavioral Scales (VABS). Subjects were evaluated with a complete medical and neurological examination, including a dermatological examination with Wood's lamp to look for signs of tuberous sclerosis and hypomelanosis of Ito. Subjects were assessed for dysmorphology and a full panel of photographs were taken for further evaluation by a clinical geneticist. Blood samples were taken from subjects and parents for DNA extraction and transformation into cell lines.

Genotyping on Oligonucleotide Arrays

Genotyping for the discovery samples (from the Central Valley of Costa Rica (“CVCR”)) was carried out at the University of California Los Angeles DNA Microarray Facility, part of the NIH Neuroscience Microarray Consortium, using manufacturer-recommended procedures for probe generation and hybridization to the NspI arrays, which contain probes for 262,264 SNPs throughout the genome at a median spacing of one SNP for every 9 kb. Analysis of this microarray data for CNVs was carried out as described previously (Nakamine et al., 2008). Analysis of the CNVs in an additional sample has been described in detail (Glessner et al., 2009).

Multiplex Ligation-Dependent Probe Amplification (MLPA)

Multiplex ligation-dependent probe amplification (MLPA) was used to confirm CNVs in the 15q11.2 region identified on SNP arrays, as described previously (Cai et al., 2008). The SALSA MLPA kit ME028 PWS/AS (MRC-Holland) was used according to the manufacturer's protocols. A total of 25 MLPA probes were included in this kit to assess genes located on this region, including TUBGCP1, CYFIP1, MKRN3, MAGEL2, NDN, SNRPN, UBE3A, ATP10A, GABRB3, OCA2, and APBA2.

Quantitative Polymerase Chain Reaction (qPCR)

In Example 2, gene expression levels of Cyfip1, Nipa1, Tubgcp5 and control genes (ActB, GusB, RPL13A and Rn18s) were determined by qPCR using the following primers:

| Gene | Forward | | Reverse | |
|---|---|---|---|---|
| Name | primers | | Primers | |
| CYFIP1 | CYFIP1 | tccatccaggagtcacagaa | CYFIP1 | agccagaaatgacctcaagc |
| | #72F | (SEQ ID NO: 7) | #72R | (SEQ ID NO: 8) |
| CYFIP1 | CYFIP1 | cagggtcacaaaactgatgaat | CYFIP1 | tcctctcagcatgacacagg |
| | #60F | (SEQ ID NO: 9) | #60R | (SEQ ID NO: 10) |
| CYFIP2 | CYFIP2 | ccagcagccatgtatcgag | CYFIP2 | tcctcgaagttcgtgtcaaa |
| | #88F | (SEQ ID NO: 11) | #88R | (SEQ ID NO: 12) |
| CYFIP2 | CYFIP2 | cagtccatccaggaatctcag | CYFIP2 | cacttccagttgctggtgaa |
| | #72F | (SEQ ID NO: 13) | #72R | (SEQ ID NO: 14) |
| NIPA1 | NIPA1 | ccccgaagtctgagagtgtg | NIPA1 | aggtagcccacaaacactgg |
| | #25F | (SEQ ID NO: 15) | #25R | (SEQ ID NO: 16) |
| NIPA1 | NIPA1 | gagttggaggagaagctgacc | NIPA1 | tcaacagcagcagcatgag |
| | #47F | (SEQ ID NO: 17) | #47R | (SEQ ID NO: 18) |
| NIPA2 | NIPA2 | ttttcattggagggagtttca | NIPA2 | atatgcatggcctccttgac |
| | #72F | (SEQ ID NO: 19) | #72R | (SEQ ID NO: 20) |
| NIPA2 | NIPA2 | tggtctgggattggctatg | NIPA2 | atatgcatggcctccttgac |
| | #67F | (SEQ ID NO: 21) | #67R | (SEQ ID NO: 22) |
| TUBGCP5 | TUBGCP5 | gctgaagaacctcaactgtgc | TUBGCP5 | gagggtatataagcttttctttctgc |
| | #27F | (SEQ ID NO: 23) | #27R | (SEQ ID NO: 24) |
| TUBGCP5 | TUBGCP5 | cctcaagtctgctgggaaga | TUBGCP5 | tccagtactgatgaactacatgctg |
| | #20F | (SEQ ID NO: 25) | #20R | (SEQ ID NO: 26) |
| ACTB | ACTB | ggatgcagaaggagattactgc | ACTB | ccaccgatccacacagagta |
| | #63F | (SEQ ID NO: 27) | #63R | (SEQ ID NO: 28) |
| GUSB | GUSB | gaggatcaacagtgcccatt | GUSB | agcctcaaaggggaggtg |
| | #31F | (SEQ ID NO: 29) | #31R | (SEQ ID NO: 30) |
| RPL13A | RPL13A | catgaggtcgggtggaagta | RPL13A | gcctgtttccgtaacctcaa |
| | #25F | (SEQ ID NO: 31) | #25R | (SEQ ID NO: 32) |
| Rn18s | Rn18s | ctcaacacgggaaacctcac | Rn18s | cgctccaccaactaagaacg |
| | #77F | (SEQ ID NO: 33) | #77R | (SEQ ID NO: 34) |

Generation of Mice with a Disruption of the Cyfip1 Gene

Mice were developed from an Omnibank (Lexicon) embryonic stem (ES) cell line that was produced by mutagenesis with a gene trap insertional vector. Briefly, an ES clone that has a trapping cassette inserted into intron 1 of the Cyfip1 gene (note that the start ATG is in exon 2) was identified. A mouse line was established from the ES cells in the 129SvEvBrd strain.

Analysis of Developmental Milestones

A systematic approach was used to assess development in the mice, following a previous described approach (Shu et al., 2005), as well as a recent detailed protocol (Heyser, 2004). Cohorts were tested beginning at 3 days in 3-day increments until the animals were 27 days old in a double-blinded manner; the tester kept track of individual pups by marking their paws with a non-toxic, low odor marker. A total of 5 litters were assessed. Each pup was observed for physical development and tested on a number of reflexes. To assess physical development, body weight was measured, while hallmarks including fur development, incisor eruption, eye opening and detachment of pinnae were observed and noted. Motor development and reflexes were monitored by appearance and/or disappearance of the righting reflex, crossed extensor reflex, and grasp reflex, and by performance in negative geotaxis, level screen test, vertical screen test, and bar holding.

### Hippocampal Slice Electrophysiology

Hippocampal slices (350 μm) were prepared from 4-6 week old heterozygous mice and their wild-type littermate controls. Slices were perfused with Ringer's solution containing (in mM): NaCl, 125.0; KCl, 2.5; MgSO4, 1.3; NaH2PO4, 1.0; NaHCO3, 26.2; CaCl2, 2.5; glucose, 11.0. The Ringer's solution was bubbled with 95% O2/5% CO2, at 32° C., during extracellular recordings (electrode solution: 3 M NaCl). Slices were maintained for 1 hr prior to establishment of a baseline of field excitatory postsynaptic potentials (fEPSPs) recorded from stratum radiatum in area CA1, evoked by stimulation of the Schaffer collateral-commissural afferents (100 μs pulses every 30 s) with bipolar tungsten electrodes placed into area CA3 (Bozdagi et al., 2000). Test stimulus intensity was adjusted to obtain fEPSPs with amplitudes that were one-half of the maximal response. The EPSP initial slope (mV/ms) was determined from the average waveform of four consecutive responses. Cycloheximide (60 μM, Sigma), dihydroxyphenylglycine (DHPG, 50 μM, Sigma), 2-methyl-6-phenylethynyl-pyridine (MPEP, 10 μM, Tocris), LY367385 (100 μM, Tocris), or rapamycin (20 nM, Enzo Life Sciences) were bath-applied for various durations as described in the Examples, below.

All experiments were performed in the presence of 100 μM 2-amino-5-phosphopentanoic acid (AP5). Paired-pulse responses were measured with interstimulus interval (ISI) of 50 ms, and are expressed as the ratio of the average responses to the second stimulation pulse (FP2) to the first stimulation pulse (FP1). LTP was induced by either a high-frequency stimulus (four trains of 100 Hz, 1 s stimulation separated by 5 min), a threshold levels of theta-burst stimulation (TBS) (5 bursts of four pulses at 100 Hz separated by 200 ms, (Lauterborn et al., 2007), or a single 100 Hz stimulation. To induce an mGluR-dependent LTD, Schaffer collaterals were stimulated by a paired-pulse low-frequency-stimulation (PP-LFS, 1 Hz for 20 min; 50 ms interstimulus interval (Huber et al., 200). DHPG-induced LTD was also used as described in the Examples, below

Data Analysis

MLPA data was analyzed as was described previously (Cai et al., 2008). qPCR data was analyzed using qBase (Hellemans et al., 2007). For additional results, data are expressed as means±SD, and statistical analyses were performed using ANOVA or Student's t-test, where P<0.05 was considered significant.

Behavioral Analysis

Cyfip1 mice were backcrossed to strain C57Bl/6Tac at least 5 times, and prepared in cohorts of 28 male animals (13 wild type and 15 heterozygotes) from 6 litters from wild type x heterozygote matings. Behavioral studies were conducted substantially as described in Nadler et al. (2004) and Elder et al. (2008). Contextual memory was tested using a contextual conditioned fear paradigm in sound-attenuated test chambers (Coulbourn Instruments) running the Freeze Frame (Actimetrics) video tracking software. Following one hour of acclimation to the test room, subjects were habituated to the test chamber with 68 dB background noise for 2 minutes. Subjects were then exposed to a series of 2 tones (20 sec, 80 dB, 2 KHz) accompanied by cue light and co-terminating shock (1 sec, 0.7 mA), separated by a 1 minute interval. Twenty-four hours later, subjects were returned to the prior test chamber without any tone or cue light, and freezing was measured for 3 minutes.

The order of behavioral testing was general observation, open-field, light dark transition, elevated zero maze, social interactions, Y-maze, Morris Water Maze, conditioned fear testing, inhibitory avoidance, and PPI.

Inhibitory avoidance was performed following the protocol published by Dölen et al. (2007) except longer cut off times were used (180 sec instead of 120 sec) during the initial training phase. Testing was performed at 6 h, 24 h and 48 h after initial training. The inhibitory avoidance box was obtained from San Diego Instruments.

For training, subjects spent 30 sec in dark chamber and were then moved to the start box in the light chamber for 90 seconds of habituation (gate closed). The gate was opened while the light remained on and latency to cross through to dark side was measured (baseline). Once the subject crossed into dark chamber, the gate was closed and the subject was subjected to 0.5 mA footshock for 2 sec. After 15 sec, the subject was returned to its home cage. Animals with baseline cross-through latencies greater than 180 sec were excluded. Subjects were tested for retention 6 h after training and for post-extinction at 24 h and 48 h after training. In each test, subjects were kept for 90 sec in lighted chamber with gate closed, the gate was opened, and cross-through latency was recorded (with a 540 sec cutoff). For the extinction phase, subjects were allowed to freely explore the dark chamber for 200 sec, with no footshock, before being returned to their home cages.

### Example 1

### Copy Number Variation (CNV) in 15q11.2 in Patients with an Autism Spectrum Diagnosis

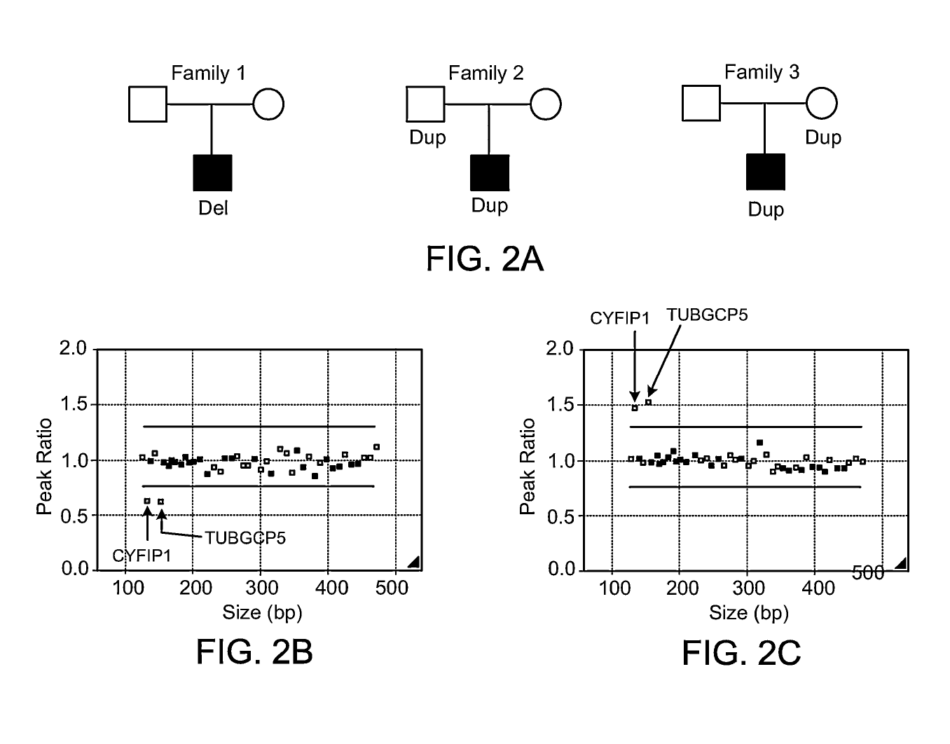

184 unrelated patients from the Central Valley of Costa Rica (“CVCR”) with an autism spectrum diagnosis were surveyed, looking for recurrent and de novo CNVs. Three families with a CNV in the 15q11.2 region were identified. Pedigrees from these three families with a 15q11.2 deletion (del) or duplication (dup) are shown in FIG. 2A. The CNV in the 15q11.2 region occurred between BP1 and BP2 (chr15:20306549-20778963, NCBI Build 36.1; see FIG. 1), and included four genes (TUBGCP5, CYFIP1, NIPA2, NIPA1, and WHAMML1) (FIG. 2B). All CNVs were confirmed with MLPA (FIGS. 2B, 2C).

### Multiplex Ligation-Dependent Amplification (MLPA) Across 15q11.2

MLPA was carried out to confirm the CNVs identified by SNP arrays. Examples from the patient with a 15q11.2 deletion (Family 1) and a patient with a 15q11.2 duplication (Family 3) are shown. All probes, except the probes between BP1-BP2 (in TUBGCP1 and CYFIP1) showed normal dosage. A copy number loss arose de novo in one case (Family 1), while two copy number gains were inherited (Family 2,3). The rate of CNVs in this cohort involving just the BP1-BP2 interval was 1.6%, with the rate of deletions (often more deleterious) being 0.5%.

To get an independent estimate rate of BP1-BP2 CNVs in ASDs, the cleaned data from a recent analysis of the AGRE cohort were surveyed, involving 1,336 patients from 785 families (Glessner et al., 2009), for CNVs involving the BP1-BP2 interval. Twelve (12) families with a CNV in the region that just included BP1-BP2 and 3 of these were deletions were identified. Hence, the rate of CNVs in this cohort involving just the BP1-BP2 interval was 1.5%, with the rate of deletions being 0.4%, in very close agreement with the results in the CVCR cohort shown in FIGS. 2A-2C.

Clinical Description of Patients with a Copy Number Variation (CNV) in 1501.2

The first patient (Family 1) was a male child, 5 years (yrs) old at the time of recruitment, born to a 21-yr-old mother and 24-yr-old father. He was the eldest of two siblings, with a 6-month-old brother. His father was healthy, with paternal history positive for mental retardation in his father's male first cousin. His mother was a healthy adult with a history of delayed language acquisition and articulations difficulties in childhood. The maternal uncle also had congenital deafness in one ear. The patient was the product of a normal full-term pregnancy. He was born with a nuchal cord but with no reported complications and Apgar scores were in the normal range (Apgar1: 8; Apgar2: 9). H is early infancy was marked by delays in some motor milestones. He held his head up at age 3 months, sat independently at 4.5 months, crawled at 7 months, stood up at 12 months, walked at 36 months, and ate independently at 41 months. Language and social milestones were also delayed with a social smile appearing around 4 months, first words at 24 months, and phrases by 54 months. He also had a problem with drooling, which improved prior to entry into preschool. Medical examinations conducted at the time of recruitment showed normal growth parameters for hearing, auditory comprehension, and EEG. Expert clinician ratings suggested a diagnosis of autistic disorder, supported by the ADOS and partially by an ADI, with scores above cutoffs on A, C, D and one point below cutoffs on B.

The second patient (Family 2) was a male child, 10 yrs old at the time of recruitment, born to a healthy 27-yr-old mother and 31-yr-old father and the eldest of two siblings, with a 6-yr-old healthy brother. Family history was positive for developmental and learning delays in the family line, including a paternal great-uncle and maternal female second cousin with congenital deafness. His mother was diagnosed with hypertension during pregnancy, and he was born via cesarean section as a result of acute fetal distress. Apgar scores at 1 and 5 min were 5 and 6, respectively. He was born at 3.2 kg and 53 cm in height. His head circumference at birth was 38 cm (90th percentile), did not normalize, and thus, he carried a diagnosis of macrocephaly at the time of recruitment. His early developmental history suggested significant motor delays including holding his head without support at 6 months, sitting at 12 months, self-feeding at 18 months, standing at 24 months, and walking at 30 months. Language/speech development was also delayed, with first words at 36 months. His history is positive for asthma and seizure activity, which started shortly before he was recruited. His physical screenings were normal. A neurological evaluation conducted at the time of recruitment found cortico-subcortical atrophy.

Patient 3 (Family 3) was a male child, 5-yrs-old at the time of recruitment, born to a 30-yr-old healthy mother and 30-yr-old father with paranoid schizophrenia. He was the youngest of three siblings, with a healthy sister and brother, ages 15 and 13 respectively at the time of the evaluation. In addition to his father's diagnosis of schizophrenia, a first cousin on the maternal side had communication and learning problem that the family considered to be autistic-like. His mother had hypertension during the 7th month of pregnancy, for which she did not receive medical interventions. His mother did receive a course of steroids to enhance fetal lung maturity. The delivery was marked by several complications including shoulder dystocia and a clavicle fracture resulting from a complicated labor requiring external help, but no forceps. Motor development during infancy progressed within normal limits. He held his head up at 2 months, sat at 7 moths, crawled at 10 months, and walked at 13 months. While first words were delayed (21 months), he used phrases by age 2. Medical and neurological exams were normal for weight, height, head circumference, and hearing. Medical reports indicate he was taking 10 mg/day of methylphenidate since age 3. Psychological testing indicated a diagnosis of autistic disorder confirmed by both the ADOS and ADI.

### Example 2

### Generation of Mice with a Disruption in the Cyfip1 Gene

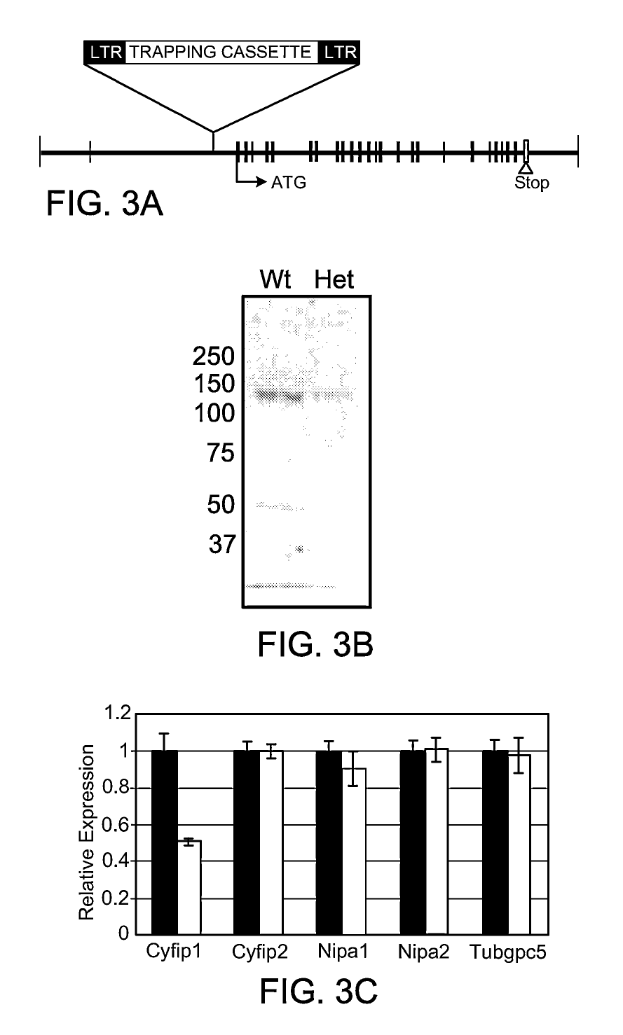

To study CYFIP1 in development, gene-trapped embryonic stem (ES) cells were used to develop mice with a disruption in Cyfip1. FIG. 3A shows the genomic structure of CYFIP1 to scale with larger horizontal boxes representing exons, and the first (ATG) and last (Stop) coding exons indicated. The site of the gene-trap insertion (identified as LTR-flanked TRAPPING CASSETTE) in intron 1 (5′ to the first coding exon), is indicated. Despite numerous attempts, mice with a disruption of both copies of Cyfip1 (knockouts) were never recovered, and even at embryonic days 4 and 5 there was no evidence for knockout embryos. These results indicated that Cyfip1 is necessary for early embryonic development.

Mice with a disruption of a single copy of Cyfip1 (heterozygotes) were obtained at expected ratios, when crossing heterozygotes inter se or crossing heterozygotes with wild-type animals. To confirm reduced expression of Cyfip1, immunoblotting and qPCR was performed, and ˜50% reduction in expression of this gene in heterozygotes was observed (FIGS. 3B, 3C). There was no compensatory change in Cyfip2, nor was there any change in expression from other genes that flank Cyfip1 (Tubgpc5, Nipa1, Nipa2), which might have been affected by the gene trap (FIG. 3C). Gene expression was normalized to a control gene: ActB, GusB, RPL13A, or Rn18S.

### Developmental Milestones in Cyfip1 Heterozygotes

To assess physical development, body weight was measured, while hallmarks including fur development, incisor eruption, eye opening and detachment of pinnae were observed and noted. Motor development and reflexes were monitored by appearance and/or disappearance of the righting reflex, crossed extensor reflex, and grasp reflex, and by performance in negative geotaxis, level screen test, vertical screen test, and bar holding. In all of measures, there was no difference between wild-type and heterozygous animals. Sensory and motor coordination was monitored by the appearance of cliff avoidance, forelimb placing, vibrissa placing, visual placing, auditory startle, tactile startle, and toe pinch. Finally, to measure emotionality, fear-induced freezing was measured after the pup was placed in a 100-ml beaker and dropped by inverting the beaker. There was no significant difference between wild-type and Cyfip1 heterozygous mice in any of these measures.

Altogether, developmental milestones in the heterozygotes were within normal limits, making them useful for further detailed studies.

### Basal Synaptic Properties in Cyfip1 Heterozygotes

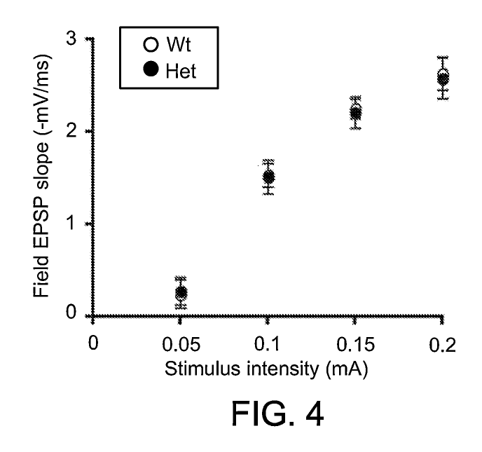

There have been extensive studies of the effect of loss of FMRP on electrophysiological properties in the hippocampus, particularly as pertains to synaptic plasticity. The association of FMRP with CYFIP1 supported similar analyses in the Cyfip1 heterozygotes. To study the basal synaptic properties in Cyfip1 heterozygotes, hippocampal slices from 4-6 weeks old wild-type (Wt) or Cyfip1 heterozygous (Het) mice were analyzed for baseline synaptic properties, determined by input/output function, which represents the relationship between stimulus intensity and the size of the field EPSP slope. Six animals per group were tested. As shown in FIG. 4, baseline synaptic properties, determined by input/output function (FIG. 4) and paired-pulse facilitation (1.26±0.06 and 1.27±0.04, in wild-type and heterozygous mice, respectively, using 6 mice per genotype, P=0.4), were not different between genotypes.

### Long-Term Potentiation in Cyfip1 Heterozygotes

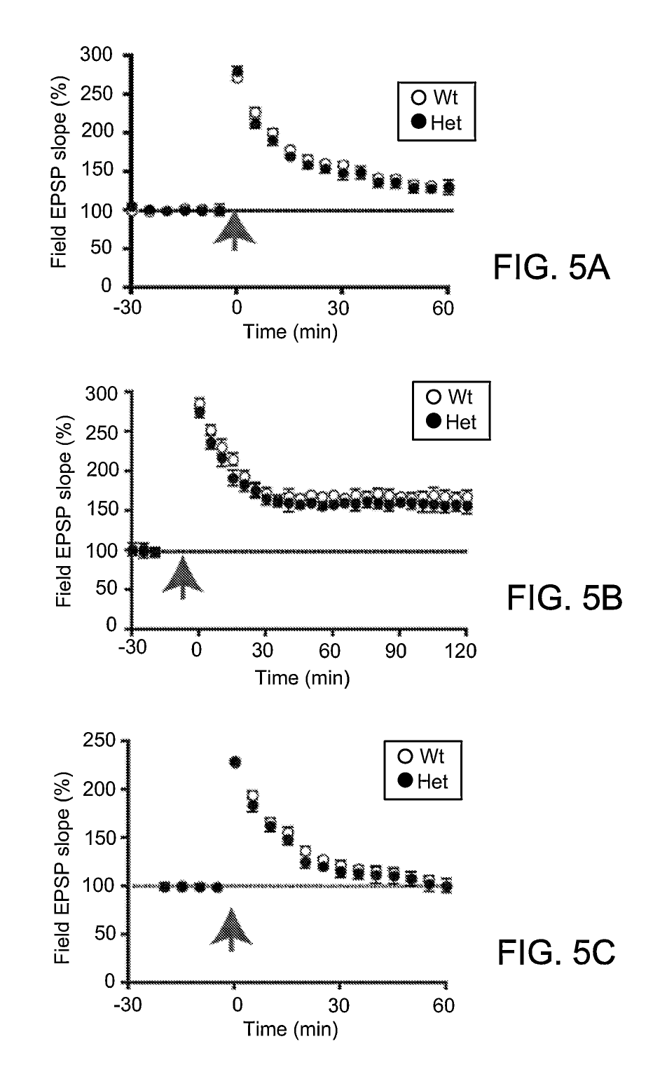

LTP is an important measure of synaptic plasticity. Prior to LTP induction, the evoked synaptic input-output relationship was examined in all acute hippocampal slices. Test pulses (100 μs duration) were collected every 30 seconds. Input-output curves were generated by setting the stimulus intensity (5-200 μA) to evoke a half-maximal slope of field EPSP at this stimulus duration. An early phase LTP (E-LTP) was induced with 100 Hz tetanic stimulation for 1 second in hippocampal slices. There were no significant differences among the genotypes with this stimulation paradigm (FIG. 5A). A protein synthesis-dependent form of LTP, induced by four trains of 100 Hz for 1 second, separated by 5 minutes, also did not differ between genotypes (FIG. 5B). Finally, induction of LTP by threshold levels of theta-burst afferent stimulation also did not demonstrate differences between genotypes (FIG. 5C).

### Long-Term Depression in Cyfip1 Heterozygotes

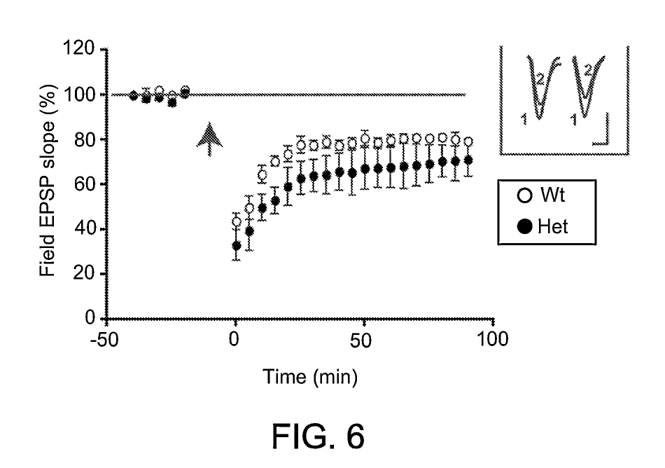

LTD is another important measure of synaptic plasticity and one that has been shown to be altered in the absence of FMRP. To examine the role of CYFIP1 in mGluR-LTD, field EPSPs were recorded at Schaffer collateral-CA1 synapses in acute hippocampal slices prepared from wild-type and Cyfip1 heterozygous mice. In slices derived from wild-type animals LTD was induced by paired-pulse low-frequency stimulation (PP-LFS) resulting in a reduction in field EPSP slope to 79.9±2.4% of baseline, while in slices derived from heterozygous animals the magnitude of the depression was significantly increased, to 67.8±8% of baseline (FIG. 6).

### Protein-Synthesis Independence of Long-Term Depression in Cyfip1 Heterozygotes

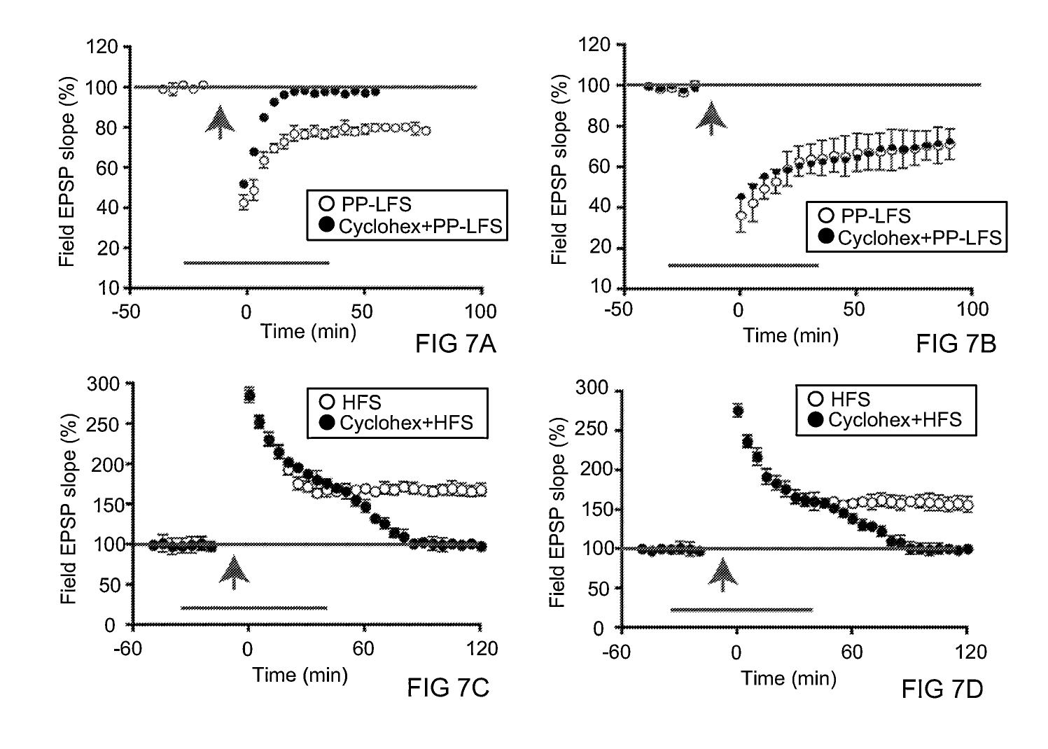

One of the most striking abnormalities in experimental synaptic plasticity observed in the absence of FMRP is that mGluR-LTD is independent of protein synthesis in these animals. As the Cyfip1 heterozygotes have enhanced LTD, similar to what is observed in the absence of FMRP, mGluR-LTD in the presence of the protein synthesis inhibitor cycloheximide was examined. Paired-pulse low frequency stimulation (PP-LFS)-induced LTD was not affected in the presence of cycloheximide (60 μM) in the Cyfip1 heterozygous animals, while the same treatment inhibited LTD in the wild-type littermate controls (FIGS. 7A, 7B). Thus, in the presence of cycloheximide, the magnitude of synaptically induced mGluR-LTD between wild-type and heterozygous mice was significantly different (at 60 min after PP-LFS, in wild-type samples incubated in the presence of cycloheximide the slope was at 98.05±2.5% of baseline while in heterozygous samples incubated in the presence of cycloheximide the slope was at 68.49±8.9% of baseline).

In contrast to the results with LTD, it was observed that the inhibition of protein synthesis-dependent LTP induced by high frequency stimulation (HFS; 4 trains of 100 Hz, 1 s stimulation separated by 5 min) with cycloheximide did not differ between genotypes (FIGS. 7C, 7D) (measuring at 120 minutes after tetanus, field EPSP slope was 168±9.4% of baseline for wild-type slices, 99±8% for wild-type slices in the presence of cycloheximide, 156.7±11.4% for heterozygous slices, and 100.8±8.2% for heterozygous slices in the presence of cycloheximide). In each panel of FIGS. 7A-7D, there were 6 animals per group.

### DHPG-Induced Long-Term Depression in Cyfip1 Heterozygotes

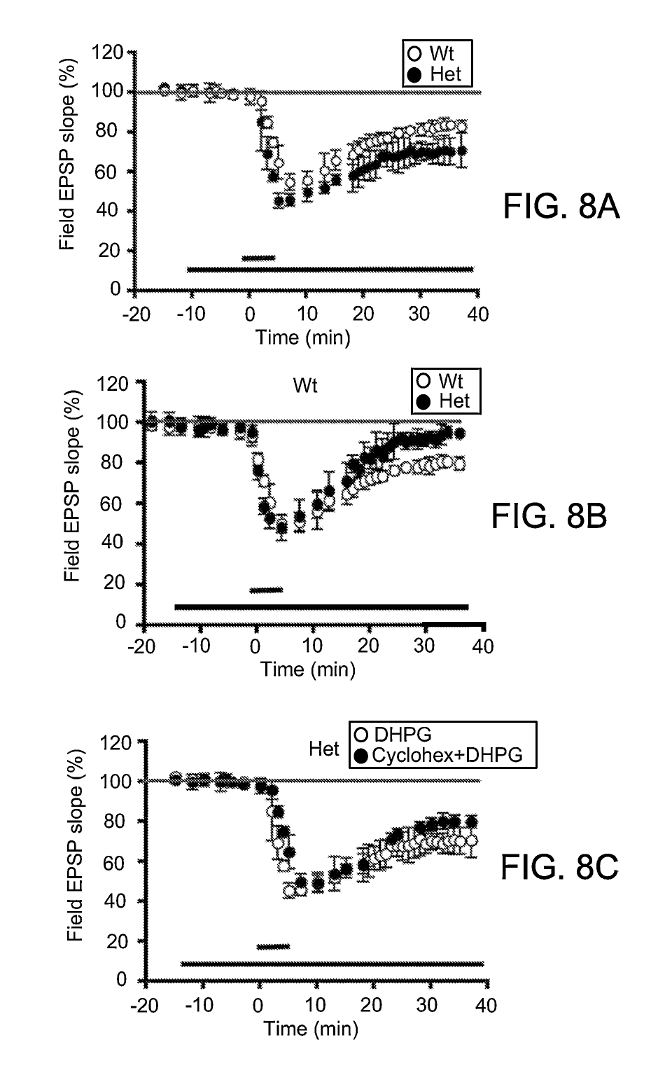

The observation that LTD, but not LTP, was independent of protein synthesis with a 50% reduction in levels of Cyfip1 was particularly intriguing. To further confirm this finding, another method to induce LTD was employed, using (RS)-3,5-dihydroxyphenylglycine (DHPG), an agonist that activates group I mGluRs. This treatment induced a depression in synaptic transmission to 81.2±3% of baseline at 30 min of application in wild-type mice (FIG. 8A). In heterozygous mice, this treatment led to significantly increased LTD (70.5±6% of baseline) (FIG. 8A). As before, the addition of 60 μM cycloheximide reduced LTD in wild-type (FIG. 8B), but not Cyfip1 heterozygous mice (FIG. 8C).

### Example 3

### The Role of the Mammalian Target of Rapamycin (mTOR) in Long-Term Depression in Cyfip1 Heterozygotes

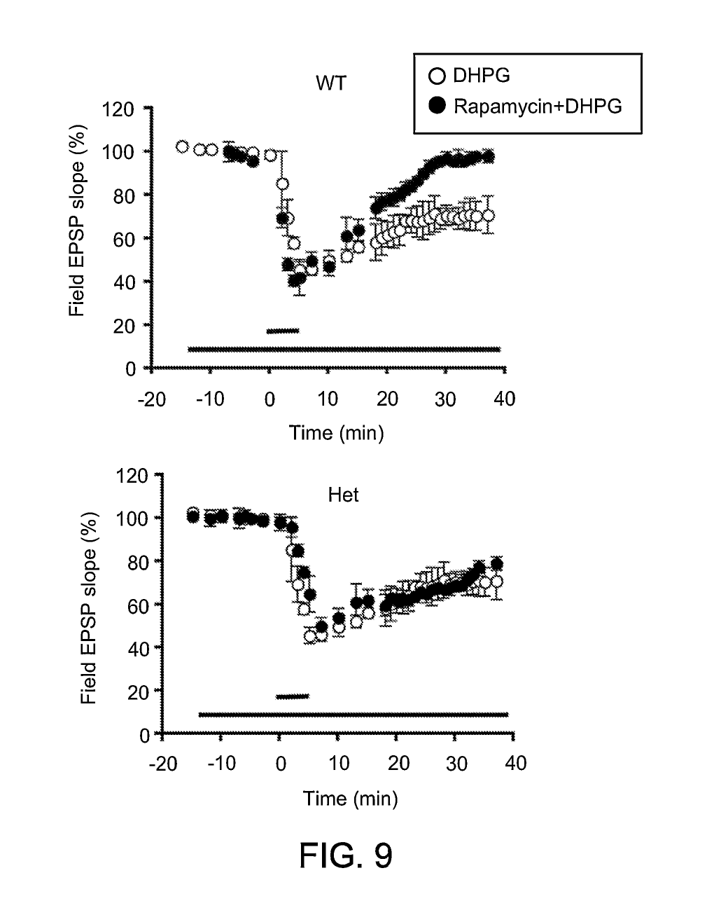

The mammalian target of rapamycin (mTOR) is an important regulator of translation in long-lasting forms of synaptic plasticity and it has been shown that DHPG-induced mGluR-LTD in hippocampal area CA1 is dependent on mTOR (see Richter and Klann, 2009). To determine whether mTOR is required for mGluR-LTD in Cyfip1 heterozygous mice, hippocampal slices were treated with DHPG in the presence of the mTOR inhibitor rapamycin. Treatment of hippocampal slices with rapamycin abolished mGluR-LTD induced by DHPG in wild-type mice but did not affect LTD in the Cyfip1 heterozygous mice (FIG. 9). Inhibition of protein synthesis-dependent LTP with rapamycin did not differ between wild-type and Cyfip1 heterozygous mice.

### Example 4

### Reversal of Enhanced LTD in Cyfip1 Heterozygotes by mGluR Antagonists

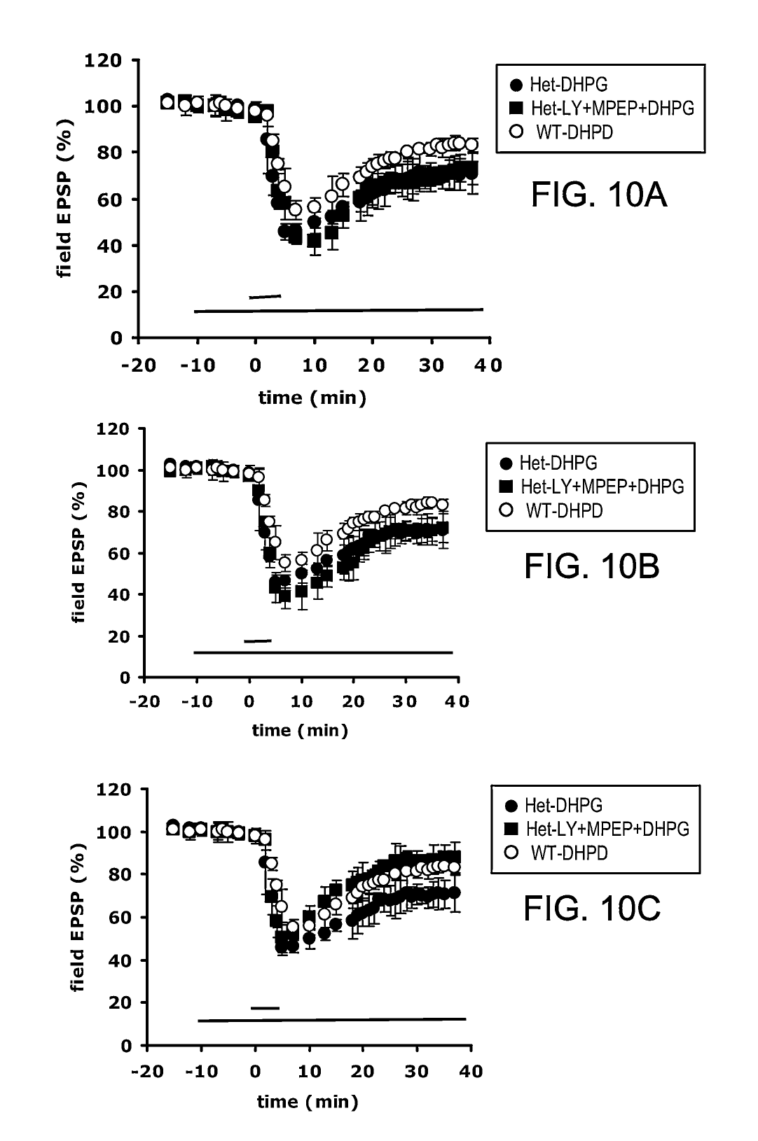

Because mGluR5 activation is essential for mGluR-LTD induction, the effect of mGluR5 blockade on DHPG-induced LTD was examined. Slices were incubated in MPEP (10 μM) and DHPG, which did not cause any change in DHPG-induced LTD in Cyfip1 heterozygotes (FIG. 10A). The effect of mGluR1 blockade on DHPG-induced LTD was also examined. Slices were incubated in LY367385 (100 μM) and DHPG, which did not cause any change in DHPG-induced LTD in Cyfip1 heterozygotes (FIG. 10B). The slices were next treated with both mGluR1 (LY367385) and mGluR5 (MPEP) antagonists. Bath application of both compounds to slices derived from Cyfip1 heterozygotes significantly decreased the magnitude of LTD in these slices to control levels (wild-type: 81.2±3% of baseline at 30 minutes of DHPG application, Cyfip1 heterozygotes, 70.5±6% of baseline, Cyfip1 heterozygotes in the presence of MPEP and LY367385, 83±5.7% of baseline, n=4, p=0.4) (FIG. 10C).

### Example 5

### Behavioral Analysis of Cyfip1 Heterozygotes

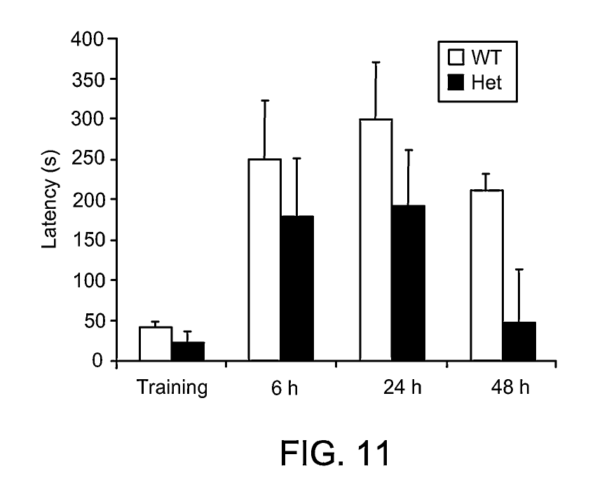

Cyfip1 heterozygous animals showed typical behaviors in assays assessing anxiety, social behaviors, and cognition. Cyfip1 heterozygotes, however, exhibited a more rapid extinction in inhibitory avoidance testing. FIG. 11, t-test P=0.027 at 48 h following training, similar to what has been described for Fmr1 knockouts. Wien et al. (2007).

### DISCUSSION

In summary, the present Examples demonstrate a role for 15q11.2 gene dosage abnormalities in ASDs, as well as in other psychiatric conditions, a conclusion consistent with prior studies in schizophrenia and in Prader-Willi and Angelman syndromes. Within this region, because of the functional and physical association of Cyfip1 with FMRP, the latter already associated with ASD phenotypes, gene dosage abnormalities in Cyfip1 are important factors for ASD phenotypes.

As shown in the present Examples, mice lacking one functional copy of Cyfip1 show enhanced LTD that is independent of protein synthesis. This observation provides a mechanism by which CYFIP1 gene changes can alter synaptic plasticity and function, and implicates shared mechanisms between FXS and loss of a functional copy of CYFIP1. Further, these Examples illustrate the present discovery that mGluR1 and mGluR5 antagonists are surprisingly useful in combination for treatment of neurological or psychiatric diseases or disorders such as FXS, schizophrenia, Prader-Willi syndrome, Angelman syndrome and ASDs, including autism.

15q11.2 in Psychiatric Conditions Including ASDs

The BP1-BP2 region of 15q11.2 has been implicated in schizophrenia and in more severe phenotypes in both PWS and AS. The current results from patients suggest that this same region might increase risk for ASDs, likely in the presence of other genetic risk factors. This conclusion is consistent with recent reports. First, a boy with a BP1-BP2 deletion was recently described: The boy presented with intellectual disability (ID), neurological disorder, developmental delay and speech impairment (Murthy et al., 2007). The deletion was inherited from a father with a similar, but milder phenotype. Not infrequently, genes associated with ID and/or developmental delay can also contribute to an ASD phenotype.

These findings received support from a more recent study where 1576 patients, ascertained for ID and/or multiple congenital abnormalities (MCA), were screened for 15q11.2 CNVs (Doornbos et al., 2009). Nine BP1-BP2 deletions were identified, two of which were de novo. Four patients with a BP1-BP2 deletion had an ASD, while 6 had general developmental delay and all had speech delay. In a study of 522 patients with ASDs from 430 families, 3 had a BP1-BP2 deletion and 2 a BP1-BP2 duplication, for a rate of about 1% for BP1-BP2 CNVs (Depienne et al., 2009). These CNVs were inherited in every case.

The study concluded that the 15q11.2 CNVs are not likely to be pathogenic. The interaction of two regions (BP1-BP2 and the adjacent regions) to enhance the severity of the phenotype in PWS and AS are consistent with a model in which the BP1-BP2 CNVs results in psychiatric phenotypes in the presence of additional genetic factors. From this perspective, the BP1-BP2 CNVs are not pathogenic in the simplest sense, but rather in the probabilistic sense, as risk factors. This interpretation is consistent with the studies in schizophrenia, where the odds ratios associated with a BP1-BP2 CNV are quite significant but still moderate. Certainly, the current estimate of 15q11.2 CNVs in controls of 1:250-1:400, as derived from these schizophrenia studies, is far lower than that observed in the studies describe herein or in those of Depienne et al.

Functional Analysis of Cyfip1

Based on the strong evidence for FMRP abnormalities in ASDs, and the evidence that CYFIP1 binds FMRP, the studies of the present Examples focused the functional analyses on CYFIP1. The data indicate that Cyfip1 heterozygous mice exhibit reduced expression of Cyfip1, which is in turn associated with an enhancement of hippocampal mGluR-LTD, without affecting hippocampal LTP, another form of synaptic plasticity, or even basal synaptic processes.

Protein synthesis is required for several different forms of synaptic plasticity, and control of protein synthesis is a critical mechanism for modulating long-term changes in neural circuits and resultant behavioral changes (Costa-Mattioli et al., 2009). Protein synthesis is required for mGluR-LTD (Huber et al., 2000). FMRP is an important regulator of translation in the brain and recently it has been shown that FMRP represses translation initiation (the rate limiting step in translation and hence an important target for regulation) via interaction with CYFIP1 (Napoli et al., 2008).

This study provides compelling evidence that CYFIP1 functions like other eukaryotic initiation factor (eIF) 4E-binding proteins (4E-BP), competing with eIF4G binding to eIF4E. Disrupting the eIF4E-eIF4G interaction inhibits translation as the bridge between the mRNA and the ribosomal preinitiation complex is lost (Costa-Mattioli et al., 2009). For canonical 4E-BP proteins, and perhaps for eIF4G, phosphorylation by an activated mTOR complex reverses the blockade on translation (Costa-Mattioli et al., 2009), a mechanism that may also occur with CYFIP1 (Napoli et al., 2008).

In the present invention, it was discovered that mGluR-LTD in Cyfip1 heterozygous mice is insensitive to inhibition of protein synthesis, demonstrating that the normal control of activity-regulated protein synthesis is lost in these mice. Previous work demonstrated that mGluR-LTD was enhanced in Fmr1 knockout mice and was unaffected by the presence of protein synthesis inhibitors (Nosyreva and Huber, 2006). The loss of the protein synthesis dependency of LTD in both these examples likely arises from a common mechanism, in which reduction of the levels of either member of a Cyfip1/Fmrp complex, disrupts the baseline suppression of local translation in the synapse.

These findings indicate that while mTOR plays a role in mGluR-LTD in wild-type mice, this mechanism is altered in Cyfip1 heterozygous mice because, as described above (Example 3), the mTOR inhibitor rapamycin only reduced mGluR-LTD in studies with the wild-type animals. Altogether, these results support dysregulation of protein synthesis in the synapse and are consistent with studies in Cyfip1 heterozygotes showing increased expression of Fmrp target genes (Napoli et al., 2008). In the present study, LTP induced by high frequency stimulation or threshold theta burst stimulation was unaltered in area CA1 in Cyfip1 heterozygotes. LTP in CA1 induced by high frequency stimulation is also known to be unaffected in Fmr1 knockout mice (Godfraind et al. 1996; Paradee et al. 1999), however, LTP elicited by threshold theta burst afferent stimulation is impaired in young adult Fmr1 knockout mice (Lauterborn et al., 2007) which is reversed by BDNF perfusion. Whether this is an age-specific effect or a difference between the two models remains to be determined.

In conclusion, the present Examples demonstrate a role for BP1-BP2 CNVs in phenotypes in patients with an ASD, as well as in patients with additional psychiatric conditions including schizophrenia, PWA, and AS. Testing for BP1-BP2 CNVs is therefore important in etiological diagnosis in these patients. Similarly, disruption of CYFIP1 (i.e. a CYFIP1 gene change), either by CNV or by mutation can contribute to etiology in some cases of ASD and other psychiatric conditions. The development of a mouse model with a loss of a functional copy of Cyfip1, as disclosed in the present Examples, provides an important resource to understand the role of this gene in psychiatric illness and in screening potential therapies.

The present invention also centers on the discovery that mGluR may be targeted with a specific combination of mGluR1 and mGluR5 antagonists. The use of the two antagonists together produced results superior to those of either alone, demonstrating that a combined approach is highly beneficial in patients having a 15q11.2 CNV, e.g., patients having schizophrenia, Prader-Willi syndrome, Angelman syndrome and ASD, and also in patients with FXS.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

### LITERATURE CITED

Douglas C Bittel, Nataliya Kibiryeva, and Merlin G Butler, “Expression of 4 genes between chromosome 15 breakpoints 1 and 2 and behavioral outcomes in Prader-Willi syndrome,” Pediatrics 118, no. 4 (October 2006): e1276-1283, doi:10.1542/peds.2006-0424.
O Bozdagi et al., “Increasing numbers of synaptic puncta during late-phase LTP: N-cadherin is synthesized, recruited to synaptic sites, and required for potentiation,” Neuron 28, no. 1 (October 2000): 245-259.
J Peter H Burbach and Bert van der Zwaag, “Contact in the genetics of autism and schizophrenia,” Trends in Neurosciences 32, no. 2 (February 2009): 69-72, doi:10.1016/j.tins.2008.11.002.
Guiqing Cai et al., “Multiplex ligation-dependent probe amplification for genetic screening in autism spectrum disorders: Efficient identification of known microduplications and identification of a novel microduplication in ASMT,” BMC Medical Genomics 1 (2008): 50, doi:10.1186/1755-8794-1-50.
J-H Chai et al., “Identification of four highly conserved genes between breakpoint hotspots BP1 and BP2 of the Prader-Willi/Angelman syndromes deletion region that have undergone evolutionary transposition mediated by flanking duplicons,” American Journal of Human Genetics 73, no. 4 (October 2003): 898-925, doi:10.1086/378816.
Edwin H Cook and Stephen W Scherer, “Copy-number variations associated with neuropsychiatric conditions,” Nature 455, no. 7215 (Oct. 16, 2008): 919-923, doi:10.1038/nature07458.
Mauro Costa-Mattioli et al., “Translational control of long-lasting synaptic plasticity and memory,” Neuron 61, no. 1 (Jan. 15, 2009): 10-26, doi:10.1016/j.neuron.2008.10.055.
Christel Depienne et al., “Screening for Genomic Rearrangements and Methylation Abnormalities of the 15q11-q13 Region in Autism Spectrum Disorders,” Biological Psychiatry (Mar. 17, 2009), doi:10.1016/j.biopsych.2009.01.025, http://www.ncbi.nlm.nih.gov/pubmed/19278672.
G. Dölen et al., “Correction of fragile X syndrome in mice,” Neuron 56, 955-962 (2007).
Marianne Doornbos et al., “Nine patients with a microdeletion 15q11.2 between breakpoints 1 and 2 of the Prader-Willi critical region, possibly associated with behavioural disturbances,” European Journal of Medical Genetics 52, no. 2-3 (June 2009): 108-115, doi:10.1016/j.ejmg.2009.03.010.
G. A. Elder et al., “Increased locomotor activity in mice lacking the low-density lipoprotein receptor,” Behav Brain Res. 191(2):256-65 (2008).
Joseph T Glessner et al., “Autism genome-wide copy number variation reveals ubiquitin and neuronal genes,” Nature 459, no. 7246 (May 28, 2009): 569-573, doi:10.1038/nature07953.
J M Godfraind et al., “Long-term potentiation in the hippocampus of fragile X knockout mice,” American Journal of Medical Genetics 64, no. 2 (Aug. 9, 1996): 246-251, doi:10.1002/(SICI)1096-8628(19960809)64:2<246::AIDAJMG2>3.0.CO; 2-S.
W Gu and J R Lupski, “CNV and nervous system diseases—what's new?,” Cytogenetic and Genome Research 123, no. 1-4 (2008): 54-64, doi:10.1159/000184692.
Jan Hellemans et al., “qBase relative quantification framework and software for management and automated analysis of real-time quantitative PCR data,” Genome Biology 8, no. 2 (2007): R19, doi:10.1186/gb-2007-8-2-r19.
Charles J Heyser, “Assessment of developmental milestones in rodents,” Current Protocols in Neuroscience/Editorial Board, Jacqueline N. Crawley . . . [et al Chapter 8 (February 2004): Unit 8.18, doi:10.1002/0471142301.ns0818s25.
19. Bernhard Horsthemke and Joseph Wagstaff, “Mechanisms of imprinting of the Prader-Willi/Angelman region,” American Journal of Medical Genetics. Part A 146A, no. 16 (Aug. 15, 2008): 2041-2052, doi:10.1002/ajmg.a.32364.
K M Huber, M S Kayser, and M F Bear, “Role for rapid dendritic protein synthesis in hippocampal mGluR-dependent long-term depression,” Science (New York, N.Y.) 288, no. 5469 (May 19, 2000): 1254-1257.
George Kirov et al., “Support for the involvement of large copy number variants in the pathogenesis of schizophrenia,” Human Molecular Genetics 18, no. 8 (Apr. 15, 2009): 1497-1503, doi:10.1093/hmg/ddp043.
M Lalande and M A Calciano, “Molecular epigenetics of Angelman syndrome,” Cellular and Molecular Life Sciences: CMLS 64, no. 7-8 (April 2007): 947-960, doi:10.1007/s00018-007-6460-0.
Julie C Lauterborn et al., “Brain-derived neurotrophic factor rescues synaptic plasticity in a mouse model of fragile X syndrome,” The Journal of Neuroscience: The Official Journal of the Society for Neuroscience 27, no. 40 (Oct. 3, 2007):10685-10694, doi:10.1523/JNEUROSCI.2624-07.2007.
S K Murthy et al., “Detection of a novel familial deletion of four genes between BP1 and BP2 of the Prader-Willi/Angelman syndrome critical region by oligoarray CGH in a child with neurological disorder and speech impairment,” Cytogenetic and Genome Research 116, no. 1-2 (2007): 135-140, doi:10.1159/000097433.
J. J. Nadler et al., “Automated apparatus for rapid quantitation of autism-like social deficits in mice,” Genes Brain Behay. 3(5):303-14 (2004).
Alisa Nakamine et al., “Duplication of 17(p11.2p11.2) in a male child with autism and severe language delay,” American Journal of Medical Genetics. Part A 146A, no. 5 (Mar. 1, 2008): 636-643, doi:10.1002/ajmg.a.31636.
Ilaria Napoli et al., “The fragile X syndrome protein represses activity-dependent translation through Cyfip1, a new 4E-BP,” Cell 134, no. 6 (Sep. 19, 2008): 1042-1054, doi:10.1016/j.cell.2008.07.031.
Elena D Nosyreva and Kimberly M Huber, “Metabotropic receptor-dependent long-term depression persists in the absence of protein synthesis in the mouse model of fragile X syndrome,” Journal of Neurophysiology 95, no. 5 (May 2006):3291-3295, doi:10.1152/jn.01316.2005.
Michael C O'Donovan, George Kirov, and Michael J Owen, “Phenotypic variations on the theme of CNVs,” Nature Genetics 40, no. 12 (December 2008):1392-1393, doi:10.1038/ng1208-1392.
W Paradee et al., “Fragile X mouse: strain effects of knockout phenotype and evidence suggesting deficient amygdala function,” Neuroscience 94, no. 1 (1999):185-192.
Joel D Richter and Eric Klann, “Making synaptic plasticity and memory last: mechanisms of translational regulation,” Genes & Development 23, no. 1 (Jan. 1, 2009): 1-11, doi:10.1101/gad.1735809.
T Sahoo et al., “Microarray based comparative genomic hybridization testing in deletion bearing patients with Angelman syndrome: genotype-phenotype correlations,” Journal of Medical Genetics 43, no. 6 (June 2006): 512-516, doi:10.1136/jmg.2005.036913.
Trilochan Sahoo et al., “Prader-Willi phenotype caused by paternal deficiency for the HBII-85 C/D box small nucleolar RNA cluster,” Nature Genetics 40, no. 6(June 2008): 719-721, doi:10.1038/ng.158.
A Schenck et al., “A highly conserved protein family interacting with the fragile X mental retardation protein (FMRP) and displaying selective interactions with FMRP-related proteins FXR1P and FXR2P,” Proceedings of the National Academy of Sciences of the United States of America 98, no. 15 (Jul. 17, 2001):8844-8849, doi:10.1073/pnas.151231598.
Weiguo Shu et al., “Altered ultrasonic vocalization in mice with a disruption in the Foxp2 gene,” Proceedings of the National Academy of Sciences of the United States of America 102, no. 27 (Jul. 5, 2005): 9643-9648, doi:10.1073/pnas.0503739102.
Hreinn Stefansson et al., “Large recurrent microdeletions associated with schizophrenia,” Nature 455, no. 7210 (Sep. 11, 2008): 232-236, doi:10.1038/nature07229.
Monica Castro Varela et al., “Phenotypic variability in Angelman syndrome: comparison among different deletion classes and between deletion and UPD subjects,” European Journal of Human Genetics: EJHG 12, no. 12 (December 2004): 987-992, doi:10.1038/sj.ejhg.5201264.

What is claimed is:

1. A method for treating a patient having a disease or disorder associated with a CYFIP1 gene change, which comprises the steps of: identifying a patient in need of such treatment and administering to said patient an effective amount for treating said disease or disorder of a composition comprising an mGluR5 antagonist and an mGluR1 antagonist.

2. The method of claim 1, wherein the disease or disorder is selected from the group consisting of an autism spectrum diagnosis (ASD), Fragile X syndrome, schizophrenia, Prader-Willi syndrome, and Angelman syndrome.

3. The method of claim 1, wherein the mGluR1 antagonist is a member selected from the group consisting of: LY367385, A 841720, LY 456236 hydrochloride, Bay 36-7620 and CPCCOEt.

4. The method of claim 1, wherein the mGluR5 antagonist is a member selected from the group consisting of 2-methyl-6-(phenylethynyl)-pyridine (MPEP), (E)-6-methyl-2-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3SR,4aRS,6SR,8aRS-6-(((4-carboxy)phenyl)methyl)-1,2,3,4,4a,5,6,7,8,8a-decahydroisoquinoline-3-carboxylic acid, [N-(3-chlorophenyl)-N′-(4,5-dihydro-1-methyl-4-oxo-1H-imidazole-2-yl)urea](Fenobam), 3-((2-Methyl-1,3-thiazol-4-yl)ethynyl)pyridine hydrochloride (MTEP hydrochloride), and 3S,4aR,6S,8aR-6-(((4-carboxy)-phenyl)methyl)-1,2,3,4,4a,5,6,7,8,8a-decahydroisoquinoline-3-carboxylic acid.

5. The method of claim 1, wherein the patient is a human.

6. The method of claim 1, which comprises administering the mGluR5 antagonist in a dose ranging from between about 0.0001 mg to about 100 mg per kilogram of body weight per day.

7. The method of claim 1, which comprises administering the mGluR1 antagonist in a dose ranging from between about 0.0001 mg to about 100 mg per kilogram of body weight per day.

8. The method of claim 1, wherein the CYFIP1 gene change is a member selected from the group consisting of a CYFIP1 duplication, a CYFIP1 deletion, a mutation in the CYFIP1 gene resulting in increased expression of CYFIP1, and a mutation in the CYFIP1 gene resulting in decreased expression of CYFIP1.

9. A method for treating a neurological or psychiatric disease or disorder, which comprises administering to a patient in need of such treatment an effective amount for treating said disease or disorder of a composition comprising an mGluR5 antagonist and an mGluR1 antagonist.

10. The method of claim 9, wherein the disease or disorder is a member selected from the group consisting of an autism spectrum diagnosis (ASD), Fragile X syndrome, schizophrenia, Prader-Willi syndrome, and Angelman syndrome.

11. The method of claim 9, wherein the mGluR1 antagonist is a member selected from the group consisting of LY367385, A 841720, LY 456236 hydrochloride, Bay 36-7620 and CPCCOEt.

12. The method of claim 9, wherein the mGluR5 antagonist is a member selected from the group consisting of 2-methyl-6-(phenylethynyl)-pyridine (MPEP), (E)-6-methyl-2-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3SR,4aRS,6SR,8aRS-6-(((4-carboxy)phenyl)methyl)-1,2,3,4,4a,5,6,7,8,8a-decahydroisoquinoline-3-carboxylic acid, [N-(3-chlorophenyl)-N′-(4,5-dihydro-1-methyl-4-oxo-1H-imidazole-2-yl)urea] (Fenobam), 3-((2-Methyl-1,3-thiazol-4-yl)ethynyl)pyridine hydrochloride (MTEP hydrochloride), and 3S,4aR,6S,8aR-6-(((4-carboxy)-phenyl)methyl)-1,2,3,4,4a,5,6,7,8,8a-decahydroisoquinoline-3-carboxylic acid.

13. The method of claim 9, wherein the patient is a human.

14. The method of claim 9, which comprises administering the mGluR5 antagonist in a dose ranging from between about 0.0001 mg to about 100 mg per kilogram of body weight per day.

15. The method of claim 9, which comprises administering the mGluR1 antagonist in a dose ranging from between about 0.0001 mg to about 100 mg per kilogram of body weight per day.

16. A pharmaceutical composition comprising (a) an mGluR5 antagonist and an mGluR1 antagonist in an effective amount for treating a disease or disorder associated with a CYFIP1 gene change; and (b) a pharmaceutically acceptable carrier or diluent.

17. The method of claim 1, which comprises intravenously administering the composition.

18. The method of claim 9, which comprises intravenously administering the composition.

19. The pharmaceutical composition of claim 16, wherein the disease or disorder is a member selected from the group consisting of an autism spectrum diagnosis (ASD), Fragile X syndrome, schizophrenia, Prader-Willi syndrome, and Angelman syndrome.

20. A pharmaceutical dosage form comprising the pharmaceutical composition of claim 16.

21. The pharmaceutical dosage form of claim 20 which is a tablet or capsule.

22. The method of claim 9, wherein said neurological or psychiatric disease or disorder is associated with a copy number variation in the 15q11.2 gene region.

23. A pharmaceutical composition comprising (a) an mGluR5 antagonist and an mGluR1 antagonist in an effective amount for treating a disease or disorder associated with a copy number variation (CNV) in the 15q11.2 gene region; and (b) a pharmaceutically acceptable carrier or diluent.

24. The pharmaceutical composition of claim 23, wherein the disease or disorder is a member selected from the group consisting of an autism spectrum diagnosis (ASD), Fragile X syndrome, schizophrenia, Prader-Willi syndrome, and Angelman syndrome.

25. A pharmaceutical dosage form comprising the pharmaceutical composition of claim 23.

26. The pharmaceutical dosage form of claim 25 which is a tablet or capsule.

27. A method for treating a patient having a disease or disorder associated with a copy number variation (CNV) in the 15q11.2 gene region, which comprises the steps of: identifying a patient in need of such treatment and administering to said patient an effective amount for treating said disease or disorder of a composition comprising an mGluR5 antagonist in combination with an mGluR1 antagonist.

28. The method of claim 27, wherein the mGlur5 antagonist is 2-methyl-6-(phenylethynyl)-pyridine (MPEP) and the mGluR1 antagonist is LY367385.

29. The method of claim 1, wherein the mGlur5 antagonist is 2-methyl-6-(phenylethynyl)-pyridine (MPEP) and the mGluR1 antagonist is LY367385.

30. The method of claim 9, wherein the mGlur5 antagonist is 2-methyl-6-(phenylethynyl)-pyridine (MPEP) and the mGluR1 antagonist is LY367385.

31. The pharmaceutical composition of claim 23, wherein the mGlur5 antagonist is 2-methyl-6-(phenylethynyl)-pyridine (MPEP) and the mGluR1 antagonist is LY367385.


SEQUENCE LISTING (34 sequences)

SEQ ID NO: 1 - DNA, 4446 bp, Homo sapiens
gagtgtttcctggcgcattcccggccagcccgagtgactcactcggccaaggaaactcccagggcccgcccaggacccccaagccgccgcggacgcagcccaggatggcggcccaggtgactctggaggacgcgctgtccaacgtggacctcctggaggagctgcccctgcccgaccagcagccctgcatcgagcccccgccatcctcgctgctctaccagccaaatttcaacactaactttgaagacagaaatgcatttgttactggcatcgcaagatacattgaacaagccaccgtccactctagcatgaacgagatgctggaggagggccaagaatatgctgtcatgctgtacacctggaggagctgctcccgggccatcccacaggtgaaatgtaacgagcagcctaacagagtggaaatctacgagaaaaccgtggaggttctggagcctgaggtcacaaaactgatgaatttcatgtacttccagagaaatgccattgagcgtttctgcggggaagtgaggcgcctgtgccatgccgagaggaggaaggacttcgtgtcagaagcctacctgatcacactgggcaaattcatcaacatgttcgctgtgctggacgagctgaagaacatgaagtgcagtgtgaagaacgaccactcagcgtacaagagggccgctcagtttttacgtaaaatggcagatccacagtccatccaggaatcgcagaatctgtccatgttcctggccaatcataacaagatcacacagtctctgcagcagcagctcgaagtgatttctggctacgaagagctcctggcagatattgtgaatctgtgtgtggattactacgagaacaggatgtatttgacgcccagtgagaaacacatgcttctcaaagtcatgggatttggtctgtacctgatggatgggagtgtcagtaacatctataagttggatgccaagaaaagaataaacttatccaaaatcgacaagtacttcaagcaactccaggtggttccgctatttggggacatgcaaatagaactggcaagatatatcaagaccagcgcccactacgaggaaaataaatctcgatggacgtgcacatcctccggcagcagccctcagtacaacatctgcgagcagatgatccagatccgcgaggaccacatgcgcttcatttcggagctggcgcgctacagcaacagcgaggtggtcacgggctcgggccgccaggaggcccagaagacggacgcggagtaccgcaagctcttcgacctggcgctgcagggcctgcagctgttgtcgcagtggagcgcgcacgtgatggaagtgtattcctggaagcttgtgcaccccaccgacaagtactccaacaaggactgccccgacagcgctgaagagtacgagcgtgccacgcgctacaactacaccagcgaggagaagtttgccctagtggaggtgatcgccatgatcaaaggcctgcaggtgctgatgggcaggatggagagcgtgttcaaccacgccatccggcacaccgtctatgccgcactgcaggacttctcccaggtgacccttagggagccgctgcggcaggccatcaagaagaagaagaacgtcatccagagtgtcctgcaggccatcaggaagaccgtgtgtgactgggagacggggcatgagcccttcaatgacccagccttgcggggcgagaaggaccccaagagcggcttcgacataaaagtaccacgccgcgccgtgggaccctccagcactcagctttacatggtgagaaccatgctagagtccctcattgcagacaaaagtggttccaagaaaaccttgagaagtagccttgaggggcccaccatattggacatagaaaaatttcatcgagagtcattcttctacactcacttgataaatttcagtgaaacgctgcagcagtgctgtgacctttcgcagctgtggttccgagagttcttcctggagctgaccatgggcaggaggatccagttccccattgagatgtcgatgccctggatcctgacggaccacatcctggagaccaaggaggcatcgatgatggagtacgtgctctactccctggacctgtacaatgacagcgcccactacgcgctcaccaggttcaacaagcagttcctgtacgacgaaattgaggccgaggtgaatctatgttttgaccaatttgtttacaagctagcagaccagatatttgcctattataaggttatggcaggaagtttgcttcttgataaacggttacgatcagaatgcaagaatcagggagccacgatccacctcccgccgtctaaccgctacgagacgctgctgaagcagaggcatgtgcagctcctcggcagatcaatagacctcaatcgtctgatcacccagcgcgtctcagcagccatgtataagtccctagaactggcgattggacgatttgaaagtgaagatttgacctccatagttgagctggatggcctgttggaaatcaaccgcatgacccacaagctgctgagccggtacctgacgctggacggcttcgacgccatgttccgggaggccaaccacaacgtgtcagcgccctacgggaggatcaccctgcacgtcttctgggagctcaactatgacttcctgcccaactactgctacaacggctctaccaaccggtttgttcggacagtgttaccattttctcaggaatttcaaagagataagcagcctaatgcacagcctcagtatctgcatggatccaaggctttgaacttggcctactccagcatttacggcagctaccggaacttcgtgggacctccacactttcaagtcatctgccggcttctcggctaccagggtatcgccgtggtcatggaggagctgctgaaggtcgtcaagagcctgctgcaaggcacaatcctgcagtacgtgaagacgctgatggaggtgatgcccaagatctgccgcctgccccggcacgagtacggctctcctggtatcctggagttcttccaccaccagctgaaggacatcgtggagtacgcagagctgaagacggtgtgcttccagaacctgcgggaggtggggaacgccatcctcttctgcctgctcatcgagcagagcctgtctttagaagaagtgtgtgacctgctgcacgcggctcctttccagaacatcttgccgcgagtccatgtgaaagagggggagagacttgatgccaaaatgaaaagactagaatcaaagtacgccccgctgcatcttgtcccactgattgaaagactggggacccctcagcaaattgccatcgcaagagagggggacctgctgacaaaggagcgcctctgctgcggcctgtccatgtttgaggtcatcctgacacggatccggagctttctggatgaccccatctggcgcgggcctctgcccagcaatggggtcatgcatgtggacgagtgtgtggagtttcacagactgtggagtgccatgcagtttgtctactgcattcccgtggggacacacgagttcacagtcgagcagtgctttggtgatgggctacactgggctggctgtatgatcatcgtacttcttgggcagcagcggcgttttgctgtgctggatttctgctaccatctacttaaagtccagaaacatgatggcaaagatgagattattaaaaatgtgcctttgaagaagatggtggagagaattcgcaagttccagattctcaatgatgagatcatcaccatcctggataagtacctgaagtcaggcgacggggagggcacgccagtggagcatgtgcgctgcttccagccgcccatccaccagtccctcgccagcagctgagggcacgcgctgcactccgtaactcaacatggcatgcctttctctccgtaaactatttagtgagatttttagggactatttttcagtatctctgtacctgttaaagggggtgcttttcgatctaaaaacttaattttataaaattgacttatttttctagactaaaattgtatatgcttttggtaattaggaactcttgagaatattggctgctgattgttgccatcacgttcctacaaaattgtttttctatgggatgttctggcagctgtgtcataaaatgctgctgggttcattcattcattccataagaaacttaataccagcaaatgcattaaatcccttgccagttaccattaactgtaactatttagcttttgtttagggatctttctgatggtcttttatgagcaatcttagttctaagtcattgttcccatcccttttttgtgtgtttcagaaaatagtgaacttgattcccctgcttccactaaatccagttgtgacaaaatctaacgtgacatcagatcgaaaggttatagaaataaaactaatgagatctaaaaaaaaaaaaaaaaaa

SEQ ID NO: 2 - DNA, 3351 bp, Homo sapiens
ccggtttaatccacaccgctgcgtctgtcctccgtcctggggctggtcttctccctcatctctgccggaggcttttaatctcttctgccactcttctggaaagtcaccagcaatgcacgctgctggctctgcaggagggtcagggggacggccctcggcctgtgcgtggtgccagccaccctccctgcctccctctgcgtgcgtcccgtgtccctgcctggggtgtgtgtgtcgagtgactaacgtctctctcctgcctctccctcctgtctgtccgtgtctagctctacctggtgcgcaccatggccgagtccttgggctctgccgagctgctcaggcagctcaagtctctgggcatggagaggctcttgcatgcggttaacacgtttctgaggcagtcgtgcacctacctaccccttttaacctttggtggtaagacatcatttgtttctcttgacgtttatggcacggaggcgaactgctccgctacaagttgttctttccccaaagcagcagcaacgtggccgcgtagacaggcacccggacccctcggggagctggtgcggggccctcccgaccagggcgtagcggagcagtccttctctcatgggctttttgagtttggcataactaatgtaccatgtatattttctcccccacaaatgtttccttggataatccagctttacatggtgagaaccatgctagagtccctcattgcagacaaaagtggttccaagaaaaccttgagaagtagccttgaggggcccaccatattggacatagaaaaatttcatcgagagtcattcttctacactcacttgataaatttcagtgaaacgctgcagcagtgctgtgacctttcgcagctgtggttccgagagttcttcctggagctgaccatgggcaggaggatccagttccccattgagatgtcgatgccctggatcctgacggaccacatcctggagaccaaggaggcatcgatgatggagtacgtgctctactccctggacctgtacaatgacagcgcccactacgcgctcaccaggttcaacaagcagttcctgtacgacgaaattgaggccgaggtgaatctatgttttgaccaatttgtttacaagctagcagaccagatatttgcctattataaggttatggcaggaagtttgcttcttgataaacggttacgatcagaatgcaagaatcagggagccacgatccacctcccgccgtctaaccgctacgagacgctgctgaagcagaggcatgtgcagctcctcggcagatcaatagacctcaatcgtctgatcacccagcgcgtctcagcagccatgtataagtccctagaactggcgattggacgatttgaaagtgaagatttgacctccatagttgagctggatggcctgttggaaatcaaccgcatgacccacaagctgctgagccggtacctgacgctggacggcttcgacgccatgttccgggaggccaaccacaacgtgtcagcgccctacgggaggatcaccctgcacgtcttctgggagctcaactatgacttcctgcccaactactgctacaacggctctaccaaccggtttgttcggacagtgttaccattttctcaggaatttcaaagagataagcagcctaatgcacagcctcagtatctgcatggatccaaggctttgaacttggcctactccagcatttacggcagctaccggaacttcgtgggacctccacactttcaagtcatctgccggcttctcggctaccagggtatcgccgtggtcatggaggagctgctgaaggtcgtcaagagcctgctgcaaggcacaatcctgcagtacgtgaagacgctgatggaggtgatgcccaagatctgccgcctgccccggcacgagtacggctctcctggtatcctggagttcttccaccaccagctgaaggacatcgtggagtacgcagagctgaagacggtgtgcttccagaacctgcgggaggtggggaacgccatcctcttctgcctgctcatcgagcagagcctgtctttagaagaagtgtgtgacctgctgcacgcggctcctttccagaacatcttgccgcgagtccatgtgaaagagggggagagacttgatgccaaaatgaaaagactagaatcaaagtacgccccgctgcatcttgtcccactgattgaaagactggggacccctcagcaaattgccatcgcaagagagggggacctgctgacaaaggagcgcctctgctgcggcctgtccatgtttgaggtcatcctgacacggatccggagctttctggatgaccccatctggcgcgggcctctgcccagcaatggggtcatgcatgtggacgagtgtgtggagtttcacagactgtggagtgccatgcagtttgtctactgcattcccgtggggacacacgagttcacagtcgagcagtgctttggtgatgggctacactgggctggctgtatgatcatcgtacttcttgggcagcagcggcgttttgctgtgctggatttctgctaccatctacttaaagtccagaaacatgatggcaaagatgagattattaaaaatgtgcctttgaagaagatggtggagagaattcgcaagttccagattctcaatgatgagatcatcaccatcctggataagtacctgaagtcaggcgacggggagggcacgccagtggagcatgtgcgctgcttccagccgcccatccaccagtccctcgccagcagctgagggcacgcgctgcactccgtaactcaacatggcatgcctttctctccgtaaactatttagtgagatttttagggactatttttcagtatctctgtacctgttaaagggggtgcttttcgatctaaaaacttaattttataaaattgacttatttttctagactaaaattgtatatgcttttggtaattaggaactcttgagaatattggctgctgattgttgccatcacgttcctacaaaattgtttttctatgggatgttctggcagctgtgtcataaaatgctgctgggttcattcattcattccataagaaacttaataccagcaaatgcattaaatcccttgccagttaccattaactgtaactatttagcttttgtttagggatctttctgatggtcttttatgagcaatcttagttctaagtcattgttcccatcccttttttgtgtgtttcagaaaatagtgaacttgattcccctgcttccactaaatccagttgtgacaaaatctaacgtgacatcagatcgaaaggttatagaaataaaactaatgagatctaaaaaaaaaaaaaaaaaa

SEQ ID NO: 3 - protein, 1253 aa, Homo sapiens
MAAQVTLEDALSNVDLLEELPLPDQQPCIEPPPSSLLYQPNFNTNFEDRNAFVTGIARYIEQATVHSSMNEMLEEGQEYAVMLYTWRSCSRAIPQVKCNEQPNRVEIYEKTVEVLEPEVTKLMNFMYFQRNAIERFCGEVRRLCHAERRKDFVSEAYLITLGKFINMFAVLDELKNMKCSVKNDHSAYKRAAQFLRKMADPQSIQESQNLSMFLANHNKITQSLQQQLEVISGYEELLADIVNLCVDYYENRMYLTPSEKHMLLKVMGFGLYLMDGSVSNIYKLDAKKRINLSKIDKYFKQLQVVPLFGDMQIELARYIKTSAHYEENKSRWTCTSSGSSPQYNICEQMIQIREDHMRFISELARYSNSEVVTGSGRQEAQKTDAEYRKLFDLALQGLQLLSQWSAHVMEVYSWKLVHPTDKYSNKDCPDSAEEYERATRYNYTSEEKFALVEVIAMIKGLQVLMGRMESVFNHAIRHTVYAALQDFSQVTLREPLRQAIKKKKNVIQSVLQAIRKTVCDWETGHEPFNDPALRGEKDPKSGFDIKVPRRAVGPSSTQLYMVRTMLESLIADKSGSKKTLRSSLEGPTILDIEKFHRESFFYTHLINFSETLQQCCDLSQLWFREFFLELTMGRRIQFPIEMSMPWILTDHILETKEASMMEYVLYSLDLYNDSAHYALTRFNKQFLYDEIEAEVNLCFDQFVYKLADQIFAYYKVMAGSLLLDKRLRSECKNQGATIHLPPSNRYETLLKQRHVQLLGRSIDLNRLITQRVSAAMYKSLELAIGRFESEDLTSIVELDGLLEINRMTHKLLSRYLTLDGFDAMFREANHNVSAPYGRITLHVFWELNYDFLPNYCYNGSTNRFVRTVLPFSQEFQRDKQPNAQPQYLHGSKALNLAYSSIYGSYRNFVGPPHFQVICRLLGYQGIAVVMEELLKVVKSLLQGTILQYVKTLMEVMPKICRLPRHEYGSPGILEFFHHQLKDIVEYAELKTVCFQNLREVGNAILFCLLIEQSLSLEEVCDLLHAAPFQNILPRVHVKEGERLDAKMKRLESKYAPLHLVPLIERLGTPQQIAIAREGDLLTKERLCCGLSMFEVILTRIRSFLDDPIWRGPLPSNGVMHVDECVEFHRLWSAMQFVYCIPVGTHEFTVEQCFGDGLHWAGCMIIVLLGQQRRFAVLDFCYHLLKVQKHDGKDEIIKNVPLKKMVERIRKFQILNDEIITILDKYLKSGDGEGTPVEHVRCFQPPIHQSLASS

SEQ ID NO: 4 - protein, 822 aa, Homo sapiens
MAESLGSAELLRQLKSLGMERLLHAVNTFLRQSCTYLPLLTFGGKTSFVSLDVYGTEANCSATSCSFPKAAATWPRRQAPGPLGELVRGPPDQGVAEQSFSHGLFEFGITNVPCIFSPPQMFPWIIQLYMVRTMLESLIADKSGSKKTLRSSLEGPTILDIEKFHRESFFYTHLINFSETLQQCCDLSQLWFREFFLELTMGRRIQFPIEMSMPWILTDHILETKEASMMEYVLYSLDLYNDSAHYALTRFNKQFLYDEIEAEVNLCFDQFVYKLADQIFAYYKVMAGSLLLDKRLRSECKNQGATIHLPPSNRYETLLKQRHVQLLGRSIDLNRLITQRVSAAMYKSLELAIGRFESEDLTSIVELDGLLEINRMTHKLLSRYLTLDGFDAMFREANHNVSAPYGRITLHVFWELNYDFLPNYCYNGSTNRFVRTVLPFSQEFQRDKQPNAQPQYLHGSKALNLAYSSIYGSYRNFVGPPHFQVICRLLGYQGIAVVMEELLKVVKSLLQGTILQYVKTLMEVMPKICRLPRHEYGSPGILEFFHHQLKDIVEYAELKTVCFQNLREVGNAILFCLLIEQSLSLEEVCDLLHAAPFQNILPRVHVKEGERLDAKMKRLESKYAPLHLVPLIERLGTPQQIAIAREGDLLTKERLCCGLSMFEVILTRIRSFLDDPIWRGPLPSNGVMHVDECVEFHRLWSAMQFVYCIPVGTHEFTVEQCFGDGLHWAGCMIIVLLGQQRRFAVLDFCYHLLKVQKHDGKDEIIKNVPLKKMVERIRKFQILNDEIITILDKYLKSGDGEGTPVEHVRCFQPPIHQSLASS

SEQ ID NO: 5 - DNA, 4112 bp, Murine
ccgccctggacaaccgaagcggcgcagaccaggatggctgcccaagtgacactagaggacgcactgtccaatgtggacctcctggaggagctgcctctgcctgaccagcagccctgcatcgagcccccaccgtcctcgctgctctatcagccaaatttcaacaccaactttgaagacagaaatgcatttgtcactggcattgcaagatacatcgaacaagcaactgtccactctagcatgaacgagatgctagaggaaggccaagagtatgctgtcatgctgtacacctggaggagctgctcccgggccatccctcaggtgaagtgcaacgagcagccgaatagagttgaaatttatgagaaaaccgtggaagtccttgaacccgaggtcacaaaactgatgaattttatgtactttcagagaaatgccatcgagcggttctgtggggaagtgagacgcctgtgtcatgctgagaggagaaaggactttgtttctgaagcctacttgatcaccctgggcaaatttatcaacatgtttggtgtgttggatgagctgaagaacatgaagtgcagtgtgaagaatgaccactctgcatataagagggctgctcagtttttacgtaaaatggcagatccacaatccatccaggagtcacagaatctgtccatgttcctggccaaccacaacaagattacacaatctctgcagcagcagcttgaggtcatttctggctatgaagagctcctggcagacatcgtgaatctgtgtgtggattactatgagaacaggatgtacttgacacccagcgaaaaacacatgcttctcaaagtcatgggatttggcctgtacctgatggatgggagtgtgagtaacatctacaaactagatgccaagaaaagaataaatttatccaaaatcgacaagtatttcaagcagctgcaggtggttccactctttggagatatgcaaatagaactggcaagatacatcaagaccagcgcacactatgaggagaataagtcccggtggacctgcgcgtcctccagcagcagcccgcagtacaacatctgtgagcagatgatccagatccgtgaggaccatatgcgtttcatctccgagctggcacgctacagcaatagcgaggtagtcacagggtctggccggcaggaagcccagaagacagatgctgagtaccggaagctcttcgatctggctctgcagggcctgcagcttctgtcgcagtggagcgctcacgcgatggaagtgtattcctggaaacttgtacatccaacagacaagtactccaacaaggactgccccgacaacgctgaggagtacgagcgagccacacgctacaactacaccactgaggagaagtttgccctggtggaggtgattgccatgatcaaaggcctgcaggtgctgatgggcaggatggagagtgtgttcaatcacgccatcagacacacggtttacgctgcgcttcaggacttctcccaggtgaccctcagggaaccactgcgacaggccatcaagaagaaaaagaacgtaatccagagtgtcctacaggccatcaggaagactgtgtgtgactgggagactgggcatgagccctttaacgacccagctctgcggggagagaaggaccctaagagtggctttgacatcaaggtgccacgccgtgctgtgggaccctccagcacgcagctttacatggtgagaactatgctagagtccctcattgcagacaaaagtggttccaagaaaaccttgagaagtagccttgaggggcccaccatattggacatagaaaaatttcatcgagaatcattcttctacactcacttgataaatttcagtgaaacactgcagcagtgctgtgacctttcccagctgtggttccgagagttcttcctggagctgaccatgggcaggaggatccagttccccattgagatgtctatgccctggattctgactgaccacatcctagagaccaaggaggcatcaatgatggagtacgtcctctactccttggacctgtacaacgacagtgcccactatgcgctcaccaagttcaacaagcagtttctctatgatgagattgaggcagaggtgaatctgtgttttgaccaatttgtttataaactagcagaccagatatttgcatattacaaggttatggcaggaagtttgcttcttgacaaaaggttacggtcagaatgcaaaaatcagggagccacgatccacctccccccatctaaccgctatgagacgctgttgaagcaaaggcacgtgcagctgctcgggagatccattgatctcaaccgtctgattacacagcgcgtttcagcagccatgtacaaatctctagaactggcaattggacggtttgaaagtgaagatttaacatcagtagtcgagctagatggactcttagaaatcaaccgtatgacacacaagctgttgagcaggtacctcacactggacagctttgatgccatgttccgggaagccaaccacaacgtgtctgcaccttatggaagaattaccctccacgtcttctgggagctaaactatgacttcttgcccaactactgctataatggctctaccaaccggtttgttcggacagtattaccattttctcaggaatttcaaagagacaaacagcctaacgcacagccccagtatttgcatggatccaaggctttgaacctagcctactcaagcatttatggcagctaccggaactttgtggggcctccgcacttccaagtcatctgccggctacttggctaccagggcattgcagtagtcatggaagagctgctgaaggttgtcaagagcctgctgcaaggcacaatcctgcagtacgtgaaaactctgatggaagtgatgcccaagatctgccggcttccgcgacatgagtacggctctcctggcatcctggagttcttccaccaccagctgaaggacattgcggagtatgcagagctgaaaaccgtatgcttccagaacctgcgggaggtgggcaatgctgttctcttctgcctgcttattgagcaaagcctgtctttagaagaagtctgtgacctgttgcatgcagctcctttccagaatatcttacctcgaatccatgtaaaagagggggagagagttgatgccaaaatgaaaagactagaatccaagtatgccccactgcaccttgtcccactgattgaaaggctgggcaccccacagcaaattgcaattgcaagagaggggcacttgctaaccaaggagcgtctctgttgtggtctgtccatgtttgaagtcatcctgacacggatccggacctttctggatgatcccatctggcgtgggcccctacccagcaatggtgtcatgcacgtggatgagtgtgtggagtttcacagactatggagtgccatgcagtttgtttactgcattcctgtagggacacacgagtttacagtggagcagtgttttggagatgggctccactgggctggctgcatgatcattgtacttcttggacagcagcggcgctttgctgtgttggatttctgctatcatcttctcaaagttcagaaacatgatggcaaagatgagatcatcaaaaatgtgccattgaagaagatggtggagaggatccgcaagttccagattctcaacgatgaaatcatcactatcctggacaagtacttgaaatctggtgatggcgagacgacacctgtggagcatgtacgctgcttccagccacccatccaccagtccctagccagtagctgagggctaagctcacagcatcccagtaactgatggcatgtttgtctttatgtaaactatattgaaatttttaggggctattttccattatgtctgaacctacttttgatctgaaagcttaactttataaaatttacttatttttctatactaaaattgtatatgcctttggaattgggaagtctgagaatacggttactagttgttgccattacggtattaacaagtattttcatgccgaatattccagcagctattataatgctaaatcactcatctgtacgagcataaccactaattgtattaaaaaaaaataaaaa

SEQ ID NO: 6 - protein, 1253 aa, Murine
MAAQVTLEDALSNVDLLEELPLPDQQPCIEPPPSSLLYQPNFNTNFEDRNAFVTGIARYIEQATVHSSMNEMLEEGQEYAVMLYTWRSCSRAIPQVKCNEQPNRVEIYEKTVEVLEPEVTKLMNFMYFQRNAIERFCGEVRRLCHAERRKDFVSEAYLITLGKFINMFGVLDELKNMKCSVKNDHSAYKRAAQFLRKMADPQSIQESQNLSMFLANHNKITQSLQQQLEVISGYEELLADIVNLCVDYYENRMYLTPSEKHMLLKVMGFGLYLMDGSVSNIYKLDAKKRINLSKIDKYFKQLQVVPLFGDMQIELARYIKTSAHYEENKSRWTCASSSSSPQYNICEQMIQIREDHMRFISELARYSNSEVVTGSGRQEAQKTDAEYRKLFDLALQGLQLLSQWSAHAMEVYSWKLVHPTDKYSNKDCPDNAEEYERATRYNYTTEEKFALVEVIAMIKGLQVLMGRMESVFNHAIRHTVYAALQDFSQVTLREPLRQAIKKKKNVIQSVLQAIRKTVCDWETGHEPFNDPALRGEKDPKSGFDIKVPRRAVGPSSTQLYMVRTMLESLIADKSGSKKTLRSSLEGPTILDIEKFHRESFFYTHLINFSETLQQCCDLSQLWFREFFLELTMGRRIQFPIEMSMPWILTDHILETKEASMMEYVLYSLDLYNDSAHYALTKFNKQFLYDEIEAEVNLCFDQFVYKLADQIFAYYKVMAGSLLLDKRLRSECKNQGATIHLPPSNRYETLLKQRHVQLLGRSIDLNRLITQRVSAAMYKSLELAIGRFESEDLTSVVELDGLLEINRMTHKLLSRYLTLDSFDAMFREANHNVSAPYGRITLHVFWELNYDFLPNYCYNGSTNRFVRTVLPFSQEFQRDKQPNAQPQYLHGSKALNLAYSSIYGSYRNFVGPPHFQVICRLLGYQGIAVVMEELLKVVKSLLQGTILQYVKTLMEVMPKICRLPRHEYGSPGILEFFHHQLKDIAEYAELKTVCFQNLREVGNAVLFCLLIEQSLSLEEVCDLLHAAPFQNILPRIHVKEGERVDAKMKRLESKYAPLHLVPLIERLGTPQQIAIAREGHLLTKERLCCGLSMFEVILTRIRTFLDDPIWRGPLPSNGVMHVDECVEFHRLWSAMQFVYCIPVGTHEFTVEQCFGDGLHWAGCMIIVLLGQQRRFAVLDFCYHLLKVQKHDGKDEIIKNVPLKKMVERIRKFQILNDEIITILDKYLKSGDGETTPVEHVRCFQPPIHQSLASS

SEQ ID NO: 7 - DNA, 20 bp, Artificial Sequence
  Synthetic oligonucleotide
tccatccaggagtcacagaa

SEQ ID NO: 8 - DNA, 20 bp, Artificial Sequence
  Synthetic oligonucleotide
agccagaaatgacctcaagc

SEQ ID NO: 9 - DNA, 22 bp, Artificial Sequence
  Synthetic oligonucleotide
cagggtcacaaaactgatgaat

SEQ ID NO: 10 - DNA, 20 bp, Artificial Sequence
  Synthetic oligonucleotide
tcctctcagcatgacacagg

SEQ ID NO: 11 - DNA, 19 bp, Artificial Sequence
  Synthetic oligonucleotide
ccagcagccatgtatcgag

SEQ ID NO: 12 - DNA, 20 bp, Artificial Sequence
  Synthetic oligonucleotide
tcctcgaagttcgtgtcaaa

SEQ ID NO: 13 - DNA, 21 bp, Artificial Sequence
  Synthetic oligonucleotide
cagtccatccaggaatctcag

SEQ ID NO: 14 - DNA, 20 bp, Artificial Sequence
  Synthetic oligonucleotide
cacttccagttgctggtgaa

SEQ ID NO: 15 - DNA, 20 bp, Artificial Sequence
  Synthetic oligonucleotide
ccccgaagtctgagagtgtg

SEQ ID NO: 16 - DNA, 20 bp, Artificial Sequence
  Synthetic oligonucleotide
aggtagcccacaaacactgg

SEQ ID NO: 17 - DNA, 21 bp, Artificial Sequence
  Synthetic oligonucleotide
gagttggaggagaagctgacc

SEQ ID NO: 18 - DNA, 19 bp, Artificial Sequence
  Synthetic oligonucleotide
tcaacagcagcagcatgag

SEQ ID NO: 19 - DNA, 21 bp, Artificial Sequence
  Synthetic oligonucleotide
ttttcattggagggagtttca

SEQ ID NO: 20 - DNA, 20 bp, Artificial Sequence
  Synthetic oligonucleotide
atatgcatggcctccttgac

SEQ ID NO: 21 - DNA, 19 bp, Artificial Sequence
  Synthetic oligonucleotide
tggtctgggattggctatg

SEQ ID NO: 22 - DNA, 20 bp, Artificial Sequence
  Synthetic oligonucleotide
atatgcatggcctccttgac

SEQ ID NO: 23 - DNA, 21 bp, Artificial Sequence
  Synthetic oligonucleotide
gctgaagaacctcaactgtgc

SEQ ID NO: 24 - DNA, 26 bp, Artificial Sequence
  Synthetic oligonucleotide
gagggtatataagcttttctttctgc

SEQ ID NO: 25 - DNA, 20 bp, Artificial Sequence
  Synthetic oligonucleotide
cctcaagtctgctgggaaga

SEQ ID NO: 26 - DNA, 25 bp, Artificial Sequence
  Synthetic oligonucleotide
tccagtactgatgaactacatgctg

SEQ ID NO: 27 - DNA, 22 bp, Artificial Sequence
  Synthetic oligonucleotide
ggatgcagaaggagattactgc

SEQ ID NO: 28 - DNA, 20 bp, Artificial Sequence
  Synthetic oligonucleotide
ccaccgatccacacagagta

SEQ ID NO: 29 - DNA, 20 bp, Artificial Sequence
  Synthetic oligonucleotide
gaggatcaacagtgcccatt

SEQ ID NO: 30 - DNA, 18 bp, Artificial Sequence
  Synthetic oligonucleotide
agcctcaaaggggaggtg

SEQ ID NO: 31 - DNA, 20 bp, Artificial Sequence
  Synthetic oligonucleotide
catgaggtcgggtggaagta

SEQ ID NO: 32 - DNA, 20 bp, Artificial Sequence
  Synthetic oligonucleotide
gcctgtttccgtaacctcaa

SEQ ID NO: 33 - DNA, 20 bp, Artificial Sequence
  Synthetic oligonucleotide
ctcaacacgggaaacctcac

SEQ ID NO: 34 - DNA, 20 bp, Artificial Sequence
  Synthetic oligonucleotide
cgctccaccaactaagaacg